(19) United States
(12) Patent Application Publication
(10) Pub. No.: US 2026/0177489 A1
(43) Pub. Date: Jun. 25, 2026

(54) IMAGING SYSTEMS AND METHODS FOR AUTOFOCUS IN AN IMAGING SYSTEM

(71) Applicant: 10x Genomics, Inc., Pleasanton, CA (US)

(72) Inventors: Anton SHUTOV, San Leandro, CA (US); Andriy TSUPRYK, Fremont, CA (US)

(21) Appl. No.: 19/422,613

(22) Filed: Dec. 17, 2025

Related U.S. Application Data
(60) Provisional application No. 63/736,541, filed on Dec. 19, 2024.

(51) Int. Cl. G01N 21/64; C12Q 1/6841

(52) U.S. Cl. CPC G01N 21/6458; C12Q 1/6841; G01N 21/6428; G01N 2021/6439; G01N 2201/0636

(57) ABSTRACT

This disclosure relates to an imaging system. The imaging system includes: an illumination dichroic mirror; a light source configured to generate illumination light and direct the illumination light to the illumination dichroic mirror. The illumination dichroic mirror is configured to direct at least a portion of the illumination light from the light source to an objective. The objective disposed to direct the illumination light to a sample positioned in the field of view of the objective. The imaging also includes an autofocus system including an autofocus light source configured to generate reference light; and an image sensor configured to receive, via the objective, a reflection of the reference light from a reflective interface in the field of view of the objective. The autofocus system is configured to direct the reference light from the autofocus light source to the illumination dichroic mirror such that the reference light is incident on the illumination dichroic mirror parallel to the illumination light, and the illumination dichroic mirror is further configured to direct at least a portion of the reference light to the objective.

### CROSS-REFERENCE TO RELATED APPLICATIONS

This application claims the benefit of U.S. Provisional Application No. 63/736,541, filed Dec. 19, 2024, which is incorporated by reference herein in its entirety, and is hereby expressly made a part of this specification.

### TECHNICAL FIELD

The disclosure relates to systems and methods for imaging a sample, and more particularly to systems and methods for autofocus, for instance in an imaging system.

### BACKGROUND

In situ detection and analysis methods are emerging from the rapidly developing field of spatial transcriptomics. The key objectives in spatial transcriptomics are to detect, quantify, and map gene activity to specific regions in a tissue sample at cellular or sub-cellular resolution. These techniques allow one to study the subcellular distribution of gene activity (as evidenced, e.g., by expressed gene transcripts), and have the potential to provide crucial insights in the fields of developmental biology, oncology, immunology, histology, etc.

Fluorescence microscopes are widely used tools that illuminate fluorescently-tagged or stained targets within a sample to image those targets with the sample. In fluorescence microscopy, fluorophores are excited by excitation light having a fluorophore-dependent excitation spectrum and then emit a fluorescence emission light having a fluorophore-dependent emission spectrum. Images of the fluorescence can be detected by a camera. Fluorescence microscopes are particularly useful in biological fields because they allow researchers to collect high-resolution images without damaging sensitive samples.

Epifluorescence microscopy, in which both the excitation light and the emission light travels through the same light path (e.g., through the same objective lens), is one implementation of a microscope used for fluorescence imaging. Transillumination microscopy, in which the excitation light illuminates the sample from the opposite side of the objective lens, is another implementation of a microscope used for fluorescence imaging.

Many fluorescence microscopes include an infinity-corrected objective to collect the emission light. Infinity corrected objectives do not form an image themselves (or, in other words, are focused at infinity—i.e., at an infinite distance or very far distance that is effectively an infinite distance) and therefore transmit the light collected from the sample as parallel, collimated beams. This type of microscope further includes a tube lens downstream of the infinity corrected objective to focus the parallel, collimated beam to a focal point. The image sensors are positioned to capture an image of the sample at or near the focal plane of the tube lens (i.e., the image of the sample is focused onto the image sensor). Such optical circuits are commonly referred to as infinity-corrected systems, where the optical path between the objective and the tube lens is referred to as the infinity space.

The infinity space provides a path of parallel (e.g. emission) light rays. Advantageously, optical components can be positioned in the infinity space without introducing aberration (e.g., spherical) or modifying the focal distance of the tube lens, making optical systems design more versatile. As such, peripheral functions of microscopes seek to make use of the infinity space.

One such peripheral function of many microscopes is provided by an autofocus module (also referred to as an autofocus system, a hardware autofocus module, a hardware autofocus system, a focus module, a focus system, a reference module, or a reference system). Some autofocus modules inject a beam of reference light into the infinity space such that the reference light passes through the objective and to a surface (or other reflective interface) in the field of view of the objective. The autofocus module also includes an image sensor (e.g., a camera) to capture a reflection of the reference light from the surface. The position of the reflection on the image sensor is dependent on the distance between the objective and the surface. By translating the surface relative to the objective, the position of the reflection changes on the image sensor. The position can be compared to a reference position on the image sensor to determine the position of the surface relative to the objective. When the position of the reflected light on the image sensor matches the reference position the surface is considered to be located at the focal plane of the objective. The autofocus system may be used in combination with the microscope focusing mechanism to maintain the surface position relative to the objective focal plane.

However, the availability of infinity space is limited. Therefore, introducing additional optical components—such as the components of a hardware autofocus module—in the infinity space can increase the cost of the instrument as a whole (e.g., by increasing the size and cost of the required tube lens). Furthermore, if the size of the infinity space can be reduced a more compact imaging system can be provided. Accordingly, a need exists to reduce the number of optical components in the infinity space. There is also a need for accurate autofocus modules that does not require increased infinity space.

### SUMMARY

This summary is provided to introduce in simplified form a selection of concepts that are further described herein. The summary is not intended to identify key or essential features of the invention.

One or more aspects of an invention are set out in the claims.

There is provided a system (e.g. an imaging system). The system comprises an illumination dichroic mirror. The system comprises a light source configured to generate illumination light and direct the illumination light to the illumination dichroic mirror. The illumination dichroic mirror is configured to direct at least a portion of the illumination light from the light source to an objective. The objective is disposed to direct the illumination light to a sample positioned in the field of view of the objective. For instance, the objective may be disposed to direct the illumination light to a focal plane of the objective, also referred to as an objective focal plane. The system also comprises an autofocus system comprising an autofocus light source configured to generate reference light. The system also comprises an image sensor configured to receive, via the objective, a reflection of the reference light from a reflective interface in the field of view of the objective. The autofocus system is configured to direct the reference light from the autofocus light source to the illumination dichroic mirror such that the reference light is incident on the illumination dichroic mirror with an optical axis parallel to the illumination light optical axis. The illumination dichroic mirror is further configured to direct at least a portion of the reference light to the objective.

Optionally, the autofocus system may further comprise a reference dichroic mirror configured to direct the reference light from the autofocus light source to the illumination dichroic mirror by reflecting at least a portion of the reference light.

Optionally, the reference dichroic mirror may be further configured to transmit at least a portion of the illumination light.

Optionally, the system may further comprise a condenser (or field lens) disposed to direct the illumination light to the illumination dichroic mirror and/or to direct the reference light from the reference dichroic mirror to the illumination dichroic mirror. Further optionally, the condenser may be disposed to direct the illumination light to travel along an optical axis of the condenser and the reference dichroic mirror may be further configured to direct the reference light to the condenser at an oblique angle to the optical axis of the condenser. The condenser is arranged to create a particular focused pattern (e.g. image of LED) on back focal plane of the objective, optionally to obtain substantially a collimated Illumination beam at the sample plane (i.e. the focal plane of the objective).

Optionally, the system may further comprise an additional image sensor configured to receive, via the objective, emission light emitted by the sample.

Optionally, the image sensor may be a reference image sensor disposed to receive the reflection of the reference light from a reflective interface in the field of view of the objective (e.g., from the objective focal plane), via the objective and the illumination dichroic mirror.

Optionally, the image sensor may be further configured to receive, via the objective, emission light emitted by the sample. Further optionally, the system may further comprise a tube lens disposed to focus the emission light from the sample onto the image sensor. Further optionally, the objective may be disposed to direct the emission light from the sample to the tube lens.

Optionally, the illumination dichroic mirror may be further configured to transmit the emission light and the reflection of the reference light from the sample to the tube lens, and the tube lens may be configured to focus the transmitted reference light onto the image sensor.

Optionally, the system may further comprise a filter disposed between the objective focal plane (e.g., a focal plane of the objective) and the image sensor. The filter may be configured to transmit at least a portion of the emission light and at least a portion of the reference light.

Optionally, the system may further comprise one or more processors which may be configured to: identify a detected position of the reflection of the reference light on the image sensor, and identify a difference between the detected position and a reference position on the image sensor.

Optionally, the reference light may be spatially coherent. Optionally, the autofocus light source may comprise a laser, an LED projector or a superluminescent diode.

Optionally, the system may further comprise an illuminator module comprising the light source and the autofocus system. Optionally, the illuminator module may be configured to change a wavelength of the illumination light by moving the light source. Optionally, the autofocus system may be configured to be stationary during movement of the light source.

Optionally, the system may further comprise one or more processors configured to detect a characteristic of the reflection of the reference light received by the image sensor to determine, based on the characteristic, a position of the sample relative to the objective focal plane. The position of the sample relative to the objective focal plane may be a position of the surface and/or the sample in the field of view of the objective. Optionally, the characteristic is a position of the reference light on the image sensor.

### BRIEF DESCRIPTION OF THE DRAWINGS

Following drawings are appended to facilitate the understanding of the invention. The drawings show embodiments, which will now be described by way of example only, where:

FIG. 1 depicts an overview of a volumetric sample imaging system and illustrates a Field of View (FOV) grid bounding the sample (e.g., hydrogel, tissue section, one or more cells, etc.) as projected onto the surface of a solid substrate supporting the sample.

FIG. 2 depicts the XZ cross-sectional view and illustrates tissue non-uniformity in the Z dimension, where the full (non-reduced) imaging volume is oversampled in the Z dimension. The objective lens focal point is positioned to acquire an image at every Z-slice in a Z-stack. An XZ image of signal distribution (bottom) demonstrates a non-uniform distribution of detected signal within the imaging volume.

FIG. 3 is an example workflow of analysis of a biological sample (e.g., a cell or tissue sample) using an opto-fluidic instrument, according to various embodiments.

FIGS. 4A-4B illustrate cross-sectional views of an optics module in an imaging system.

FIG. 5 depicts a computing node according to some embodiments disclosed herein.

FIG. 6 depicts an image system suitable for fluorescence microscopy that includes an autofocus module.

FIGS. 7A-7C depict image systems suitable for fluorescence microscopy according to the present disclosure.

FIG. 8 depicts a method for focusing an imaging system.

FIG. 9 depicts the relationship of the distance between an objective and the surface in the z-direction and the position of a reflection of a reference light beam on an image sensor in imaging systems according to the present disclosure.

In the figures, elements and steps having the same or similar reference numeral have the same or similar attributes or description, unless explicitly stated otherwise.

The patent or application file contains at least one drawing executed in colour. Copies of this patent or patent application publication with colour drawing(s) will be provided by the Office upon request and payment of the necessary fee.

### DETAILED DESCRIPTION

### Overview

The following overview is provided to introduce in simplified form a selection of concepts that are further described herein. The overview is not intended to identify only key or essential features of the invention.

The present disclosure relates to an imaging system with an illumination dichroic mirror, a light source (also referred to herein as an illumination light source), an objective, a movable surface, an autofocus system, and an image sensor. The autofocus system includes an autofocus light source configured to generate reference light and direct the reference light from the autofocus light source to the illumination dichroic mirror. In some embodiments, the reference light is incident on the illumination dichroic mirror with an optical axis parallel that of the illumination light from the light source. The illumination dichroic mirror is configured to direct at least a portion of the reference light to the objective. As such, the reference light is directed by the objective to an objective focal plane through which the surface is movable. In some embodiments, the illumination dichroic mirror is configured to direct at least a portion of the reference light to the objective by reflection. As the illumination dichroic mirror directs the reference light to the objective, the imaging system of the present disclosure does not require a dedicated reference dichroic mirror in the imaging system's infinity space to direct the reference light to the objective.

Accordingly, compared to previous imaging systems, the imaging system of the present disclosure increases infinity space availability for additional peripheral functions and reduces the imaging system cost. In addition, directing the reference light to the objective via the same illumination dichroic mirror that directs at least a portion of the illumination light increases the optical path shared between the reference light and the illumination light. As a result of increasing the shared optical path, changes in operational behaviour of the components—due to, for instance, thermal drift—in the optical path (such as the illumination dichroic mirror or the objective) affect both the illumination light and the reference light. As a result, the autofocus system of the present disclosure has increased accuracy during prolonged use because operational behaviour changes affect the focal distance of the reference light in the same way as of the illumination light and thus are accounted for in the autofocus system. As an example, operational behaviour changes may include changes to the alignment of, orientation of, focal lengths of, or aberration introduced by the optical components of the respective imaging systems.

Embodiments are directed to addressing these and other problems associated with infinity space availability and operational behaviour changes in imaging systems with autofocus systems.

In some embodiments, the image sensor is configured to receive both emission light and the reflection of the reference light. In some of these embodiments the imaging system further includes a tube lens dispose to focus the emission light from the surface onto the emission-receiving image sensor. As such, in these embodiments, the imaging path shared between emission light emitted by a sample on the surface and the reflection of the reference light from the sample is increased. The imaging path passes from the surface, through any combination of the objective, the illumination dichroic mirror and/or the tube lens, and to the image sensor. Accordingly, changes in operational behaviour of the components—due to, for instance, thermal drift—in the imaging path affect both the emission light and the reflection of the reference light. As a result, the autofocus system of the present disclosure has increased accuracy during prolonged use because operational behaviour changes affect the optical characteristics of the emission light on the image sensor similarly as to the reflection of the reference light. In addition, using one image sensor for both the reference light and the emission light, reduces the cost of manufacture for embodiments of the imaging systems according to present disclosure.

In some embodiments, the image sensor is a reference image sensor. The reference image sensor is disposed to receive a reflection of the reference light from the surface, via the objective and the illumination dichroic mirror. In some of these embodiments, the system further includes an additional image sensor configured to receive the illumination light and/or emission light from a sample on the surface. These embodiments retain the modularity of the autofocus system because the reference image sensor is separate from the additional image sensor for receiving the illumination light. As such, these embodiments can be more easily installed into imaging systems, while maintaining the capability to account for operational behaviour changes of optical components as described herein.

In the following, embodiments will be discussed in more detail with reference to the appended drawings. It should be understood, however, that the drawings are not intended to limit the present disclosure to the subject-matter depicted in the drawings. The embodiments described with reference to the drawings can be understood in isolation from, as well as in the context of, the concepts set out in the claims, summary and/or overview of the present disclosure.

### Volumetric Sample Imaging Systems

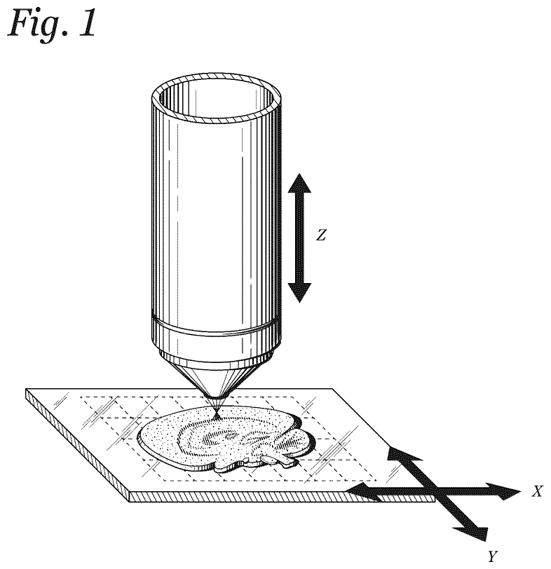

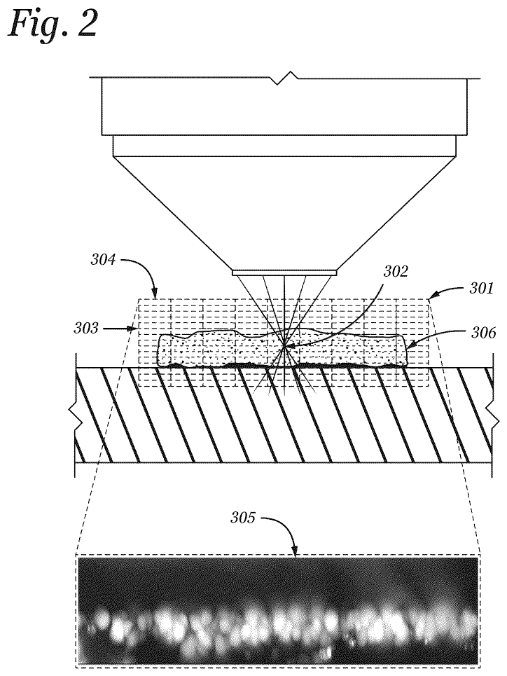

In volumetric sample imaging systems (e.g., an optofluidic instrument), a z-stack of images is obtained for each Field of View (FOV) of the objective (FIG. 1). For such automated, high-throughput tissue imaging applications, automatically identifying relevant regions—those regions that contain target molecules such as nucleic acids or proteins—can be challenging as distribution of tissue is non-uniform in many biological samples (FIG. 2). FIG. 2 depicts the XZ cross-sectional view and illustrates tissue non-uniformity in the Z dimension in a tissue section 306, where the full (non-reduced) imaging volume 301 is oversampled in the Z dimension. The objective lens focal point 302 is positioned to acquire an image at every Z-slice 303 in a Z-stack 304. An XZ image of signal distribution 305 (bottom) demonstrates a non-uniform distribution of detected signal within the imaging volume. The data extracted from the detection and analysis methods disclosed herein (e.g., in situ detection and analysis of target analytes, such as SBS, SBL, SBH; and in situ hybridization techniques, such as smFISH and MERFISH) include the relative coordinates within a field of view (FOV) and provides intricate information regarding tissue organization.

In general, the systems and methods described herein use any suitable method to generate contrast of a sample against a background (e.g., illumination of a sample via bright field imaging, illumination of a sample via fluorescent imaging, inducing autofluorescence within the sample, adding contrast to the sample with one or more stains, etc.)

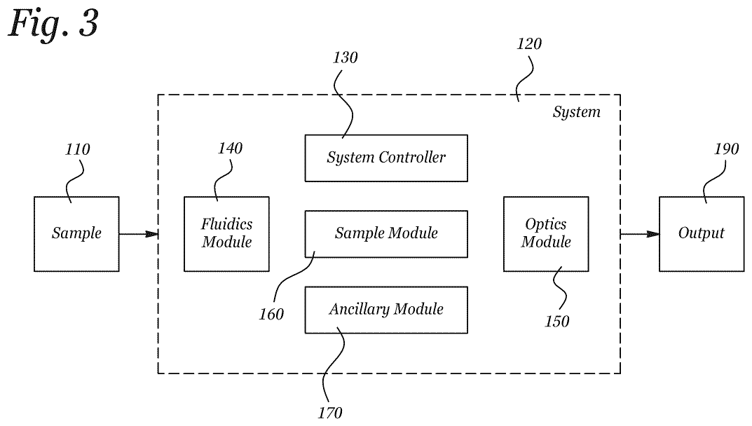

FIG. 3 shows an example workflow of analysis of a biological sample 110 (e.g., cell or tissue sample) using an opto-fluidic instrument 120, according to various embodiments. In various embodiments, the sample 110 can be a biological sample (e.g., a tissue) that includes molecules such as DNA, RNA, proteins, antibodies, etc. For example, the sample 110 can be a sectioned tissue that is treated to access the RNA thereof for labelling with circularizable DNA probes. Ligation of the probes may generate a circular DNA probe which can be enzymatically amplified and bound with fluorescent oligonucleotides, which can create a bright signal that is convenient to image and has a high signal-to-noise ratio.

In various embodiments, the sample 110 may be placed in the opto-fluidic instrument 120 for analysis and detection of the molecules in the sample 110. In various embodiments, the opto-fluidic instrument 120 can be a system configured to facilitate the experimental conditions conducive for the detection of the target molecules. For example, the opto-fluidic instrument 120 can include a fluidics module 140, an optics module 150, a sample module 160, and an ancillary module 170, and these modules may be operated by a system controller 130 to create the experimental conditions for the probing of the molecules in the sample 110 by selected probes (e.g., circularizable DNA probes), as well as to facilitate the imaging of the probed sample (e.g., by an imaging system of the optics module 150). In various embodiments, the various modules of the opto-fluidic instrument 120 may be separate components in communication with each other, or at least some of them may be integrated together.

In various embodiments, the sample module 160 may be configured to receive the sample 110 into the opto-fluidic instrument 120. For instance, the sample module 160 may include a sample interface module (SIM) that is configured to receive a sample device (e.g., cassette) onto which the sample 110 can be deposited. That is, the sample 110 may be placed in the opto-fluidic instrument 120 by depositing the sample 110 (e.g., the sectioned tissue) on a sample device that is then inserted into the SIM of the sample module 160. In some instances, the sample module 160 may also include an X-Y stage onto which the SIM is mounted. The X-Y stage may be configured to move the SIM mounted thereon (e.g., and as such the sample device containing the sample 110 inserted therein) in perpendicular directions along the two-dimensional (2D) plane of the opto-fluidic instrument 120.

The experimental conditions that are conducive for the detection of the molecules in the sample 110 may depend on the target molecule detection technique that is employed by the opto-fluidic instrument 120. For example, in various embodiments, the opto-fluidic instrument 120 can be a system that is configured to detect molecules in the sample 110 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 110 using reagents such as washing/stripping reagents, hybridizing reagents, etc., and such reagents may be provided by the fluidics module 140.

In various embodiments, the fluidics module 140 may include one or more components that may be used for storing the reagents, as well as for transporting said reagents to and from the sample device containing the sample 110. For example, the fluidics module 140 may include reservoirs configured to store the reagents, as well as a waste container configured for collecting the reagents (e.g., and other waste) after use by the opto-fluidic instrument 120 to analyze and detect the molecules of the sample 110. Further, the fluidics module 140 may also include pumps, tubes, pipettes, etc., that are configured to facilitate the transport of the reagent to the sample device (e.g., and as such the sample 110). For instance, the fluidics module 140 may include pumps (“reagent pumps”) that are configured to pump washing/stripping reagents to the sample device for use in washing/stripping the sample 110 (e.g., as well as other washing functions such as washing an objective lens of the imaging system of the optics module 150).

In various embodiments, the ancillary module 170 can be a cooling system of the opto-fluidic instrument 120, and the cooling system may include a network of coolant-carrying tubes that are configured to transport coolants to various modules of the opto-fluidic instrument 120 for regulating the temperatures thereof. In such cases, the fluidics module 140 may include coolant reservoirs for storing the coolants and pumps (e.g., “coolant pumps”) for generating a pressure differential, thereby forcing the coolants to flow from the reservoirs to the various modules of the opto-fluidic instrument 120 via the coolant-carrying tubes. In some instances, the fluidics module 140 may include returning coolant reservoirs that may be configured to receive and store returning coolants, i.e., heated coolants flowing back into the returning coolant reservoirs after absorbing heat discharged by the various modules of the opto-fluidic instrument 120. In such cases, the fluidics module 140 may also include cooling fans that are configured to force air (e.g., cool and/or ambient air) into the returning coolant reservoirs to cool the heated coolants stored therein. In some instances, the fluidics module 140 may also include cooling fans that are configured to force air directly into a component of the opto-fluidic instrument 120 so as to cool said component. For example, the fluidics module 140 may include cooling fans that are configured to direct cool or ambient air into the system controller 130 to cool the same.

As discussed above, the opto-fluidic instrument 120 may include an optics module 150 which include the various optical components of the opto-fluidic instrument 120, such as but not limited to a camera, an illumination module (e.g., light source such as LEDs), an objective lens, and/or the like. The optics module 150 may include a fluorescence imaging system that is configured to image the fluorescence emitted by the probes (e.g., oligonucleotides) in the sample 110 after the probes are excited by light from the illumination module of the optics module 150.

In some instances, the optics module 150 may also include an optical frame onto which the camera, the illumination module, and/or the X-Y stage of the sample module 160 may be mounted.

In various embodiments, the system controller 130 may be configured to control the operations of the opto-fluidic instrument 120 (e.g., and the operations of one or more modules thereof). In some instances, the system controller 130 may take various forms, including a processor, a single computer (or computer system), or multiple computers in communication with each other. In various embodiments, the system controller 130 may be communicatively coupled with data storage, set of input devices, display system, or a combination thereof. In some cases, some or all of these components may be considered to be part of or otherwise integrated with the system controller 130, may be separate components in communication with each other, or may be integrated together. In other examples, the system controller 130 can be, or may be in communication with, a cloud computing platform.

In various embodiments, the opto-fluidic instrument 120 may analyze the sample 110 and may generate the output 190 that includes indications of the presence of the target molecules in the sample 110. For instance, with respect to the example embodiment discussed above where the opto-fluidic instrument 120 employs a hybridization technique for detecting molecules, the opto-fluidic instrument 120 may cause the sample 110 to undergo successive rounds of fluorescent probe hybridization (using two or more sets of fluorescent probes, where each set of fluorescent probes is excited by a different color channel) and be imaged to detect target molecules in the probed sample 110. In such cases, the output 190 may include optical signatures (e.g., a codeword) specific to each gene, which allow the identification of the target molecules.

In some instances, an assembly for transilluminating a substrate can include a sample carrier device (e.g., a microfluidic chip or glass slide), a thermal control module configured to control the temperature of the sample carrier device (e.g., a thermoelectric module), and a light source configured to illuminate the sample carrier device. In some instances, the assembly includes a heat exchanger (e.g., a fluid block having a cooling fluid flowing therethrough). In some instances, an assembly for transilluminating can include sample carrier device (e.g., a sample substrate), an optically transparent substrate, a light source configured to illuminate the optically transparent substrate, a light scattering layer configured to scatter light from the light source, and/or a thermal control module configured to control the temperature of the sample carrier device and/or optically transparent substrate.

In some embodiments, the sample carrier device (e.g., a cassette) can be configured to receive a sample. In some embodiments, the sample carrier device can include one or more microfluidic channels, e.g., sample chambers or microfluidic channels etched into a planar substrate or chambers within a flow cell or microfluidic device.

A sample carrier device for the systems disclosed herein can include, but is not limited to, a substrate configured to receive a sample, a microscope slide and/or an adapter configured to mount microscope slides (with or without coverslips) on a microscope stage or automated stage (e.g., an automated translation or rotational stage), a substrate, and/or an adapter configured to mount slides on a microscope stage or automated stage, a substrate comprising etched sample containment chambers (e.g., chambers open to the environment) and/or an adapter configured to mount such substrates on a microscope stage or automated stage, a flow cell and/or an adapter configured to mount flow cells on a microscope stage or automated stage, or a microfluidic device and/or an adapter configured to mount microfluidic devices on a microscope stage or automated stage. In some embodiments, the sample carrier device further includes a cassette configured to secure a substrate (e.g., a glass slide). In some embodiments, the cassette includes two or more components (e.g., a top half and a bottom half) into which the substrate is secured.

In some instances, the one or more sample carrier devices can be designed for performing a variety of chemical analysis, biochemical analysis, nucleic acid analysis, cell analysis, or tissue analysis applications. In some instances, for example, the sample carrier device (e.g., flow cells and microfluidic devices) may comprise a sample, e.g., a tissue sample. In some instances, the sample carrier device (e.g., flow cells and microfluidic devices) may comprise a sample, e.g., a tissue sample, placed in contact with, e.g., a substrate (e.g., a surface of the flow cell or microfluidic device).

The sample carrier devices for the disclosed systems (e.g., microscope slides, substrates comprising one or more etched microfluidic channel, flow cells or microfluidic devices comprising one or more microfluidic channels, etc.) can be fabricated from any of a variety of materials known to those of skill in the art including, but not limited to, glass (e.g., borosilicate glass, soda lime glass, etc.), fused silica (quartz), silicon, polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), polydimethylsiloxane (PDMS), etc.), polyetherimide (PEI) and perfluoroelastomer (FFKM) as more chemically inert alternatives, or any combination thereof. FFKM is also known as Kalrez.

The one or more materials used to fabricate sample carrier devices for the disclosed systems (e.g., substrates configured to receive a sample, microscope slides, substrates comprising one or more etched microfluidic channels, flow cells or microfluidic devices comprising one or more microfluidic channels or sample chambers, etc.) can be optically transparent to facilitate use with spectroscopic or imaging-based detection techniques. In some instances, the entire sample carrier device can be optically transparent. Alternatively, in some instances, only a portion of the sample carrier device (e.g., an optically transparent “window”) can be optically transparent.

The sample carrier devices for the disclosed systems (e.g., substrates configured to receive a sample, microscope slides, substrates comprising one or more etched microfluidic channels, flow cells or microfluidic devices comprising one or more microfluidic channels or sample chambers, etc.) can be fabricated using any of a variety of techniques known to those of skill in the art, where the choice of fabrication technique is often dependent on the choice of material used, and vice versa. Examples of suitable sample carrier device fabrication techniques include, but are not limited to, extrusion, drawing, precision computer numerical control (CNC) machining and boring, laser photoablation, photolithography in combination with wet chemical etching, deep reactive ion etching (DRIE), micro-molding, embossing, 3D-printing, thermal bonding, adhesive bonding, anodic bonding, and the like (see, e.g., Gale, et al. (2018), “A Review of Current Methods in Microfluidic Device Fabrication and Future Commercialization Prospects”, Inventions 3, 60, 1-25, which is hereby incorporated by reference in its entirety).

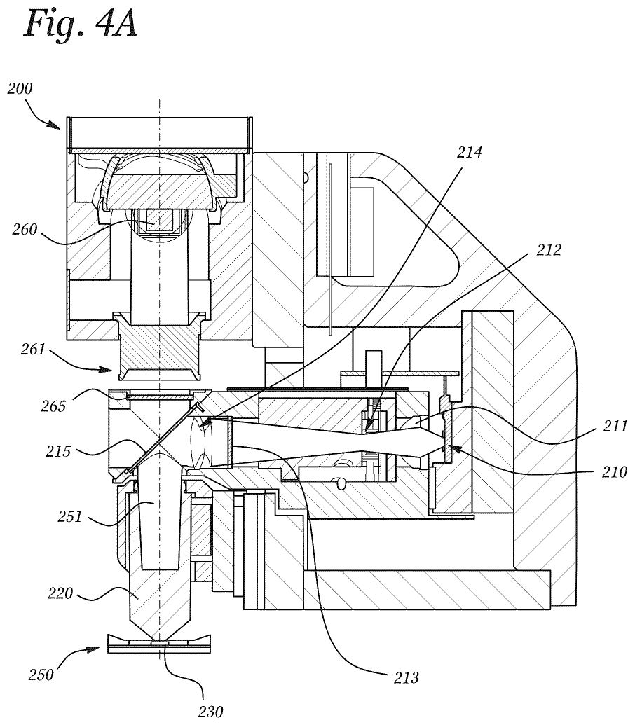

FIG. 4A illustrates a cross-sectional view of an optics module 200 in a comparative imaging system. One or more illumination sources 210, e.g., one or more light emitting diodes (LEDs), provides light through one or more optical components and an objective lens 220 to thereby illuminate a sample 230 in a sample holder 250. In various embodiments, the optical components include a collimator 211. In various embodiments, the optical components include a field stop 212. In various embodiments, the optical components include one or more excitation filters 213. In various embodiments, the one or more excitation filters 213 are configured to filter light from the illumination source(s) 210 for a predetermined range of wavelengths (e.g., each filter has one or more blocking band(s) and/or transmission band(s) that may be different or may overlap at least in part) and each excitation filter 213 is aligned with appropriate illumination sources (e.g., blue LEDs, green LEDs, yellow LEDs, red LEDs, ultraviolet LEDs, etc.). In various embodiments, the optical components include a condenser 214. In various embodiments, the optical components include a beam splitter 215. An optical axis 251 is illustrated extending through the center of the optical surfaces in the objective lens 220 and its path includes an image plane, a focal plane, and input/output pupils (illustrated in FIG. 4B—also showing a comparative imaging system 200 comprising an image plane 401, an object plane 402, a pupil 403, a 1.0 NA 20× objective 404, a 26.5 mm FN tube lens 405 and a small pixel, large sensor, fast readout camera 406).

A sensor array 260 (e.g., CMOS sensor) receives light signals from the sample 250. In various embodiments, the optical components include one or more emission filters 265. In various embodiments, the one or more emission filters 265 are configured to filter light from the sample (e.g., emitted from one or more fluorophores, autofluorescence, etc.) for a predetermined range of wavelengths (e.g., each filter has one or more blocking band(s) and/or transmission band(s) that may be different or may overlap at least in part). In various embodiments, the emission filters 265 align (e.g., via motorized translation) with optics and/or the sensor array. In various embodiments, the sample 230 is probed with fluorescent probes configured to bind to a target (e.g., DNA or RNA) that, when illuminated with a particular wavelength (or range of wavelengths) of light, emit light signals that can be detected by the sensor array 260. In various embodiments, the sample 230 is repeatedly probed with two or more (e.g., two, three, four, five, six, etc.) different sets of probes. In various embodiments, each set of probes corresponds to a specific color (e.g., blue, green, yellow, or red) such that, when illuminated by that color, probes bound to a target emit light signals. In some embodiments, the sensor array 260 is aligned with the optical axis 251 of the objective lens 220 (i.e., the optical axis of the camera is coincident with and parallel to the optical axis of the objective lens 220). In various embodiments, the sensor array 260 is positioned perpendicularly to the objective lens 220 (i.e., the optical axis of the camera is perpendicular to and intersects the optical axis of the objective lens 220). In various embodiments, a tube lens 261 is mounted in the optical path to focus light on the sensor array 260 thereby allowing for image formation with infinity-corrected objectives. Descriptions of optical modules and illumination assemblies for use in opto-fluidic instruments can be found in U.S. provisional patent application No. 63/427,282, filed on Nov. 22, 2022, titled “Systems and Methods for Illuminating a Sample” and U.S. provisional patent application No. 63/427,360, file on Nov. 22, 2022, titled “Systems and Methods for Imaging Samples,” each of which is incorporated by reference in its entirety.

In various embodiments, the sample is illuminated with one or more wavelengths configured to induce fluorescence in the sample. In various embodiments, the sample is probed during one or more probing cycles with one or more fluorescent probes configured to bind to one or more target analytes. In various embodiments, the one or more wavelengths are selected to induce fluorescence in a subset of the one or more fluorescent probes. In various embodiments, each probing cycle includes illumination with two or more (e.g., four) colors of light. In various embodiments, the sample is treated with a fluorescent stain configured to illuminate one or more structures within the sample. In various embodiments, the sample is contacted with a nuclear stain. In various embodiments, the sample is contacted with 4′,6-diamidino-2-phenylindole (“DAPI”) configured to bind to adenine-thymine-rich regions in DNA. In various embodiments, illumination of the sample causes autofluorescence of the sample. In various embodiments, autofluorescence is the natural emission of light by biological structures when they have absorbed light, and may be used to distinguish the light originating from artificially added fluorescent markers. In various embodiments, fluorescence of the sample through fluorescent probes, autofluorescence, and/or a fluorescent stain can be used with the methods described herein to determine one or more focus metrics of a tissue sample.

In various embodiments, the sample is illuminated via edge lighting or transillumination along one or more edges of the sample and/or sample substrate. In various embodiments, the edge lighting provides dark-field illumination of the sample. In various embodiments, edge lighting is provided by one or more light sources positioned to provide light substantially perpendicular to a normal of the substrate surface on which the sample is disposed. In various embodiments, the substrate is a glass slide. In various embodiments, the substrate is configured as a wave guide to thereby guide light emitted from the edge lighting towards the sample. In various embodiments, illumination of the sample via edge lighting can be used with the methods described herein to determine one or more focus metrics of a tissue sample.

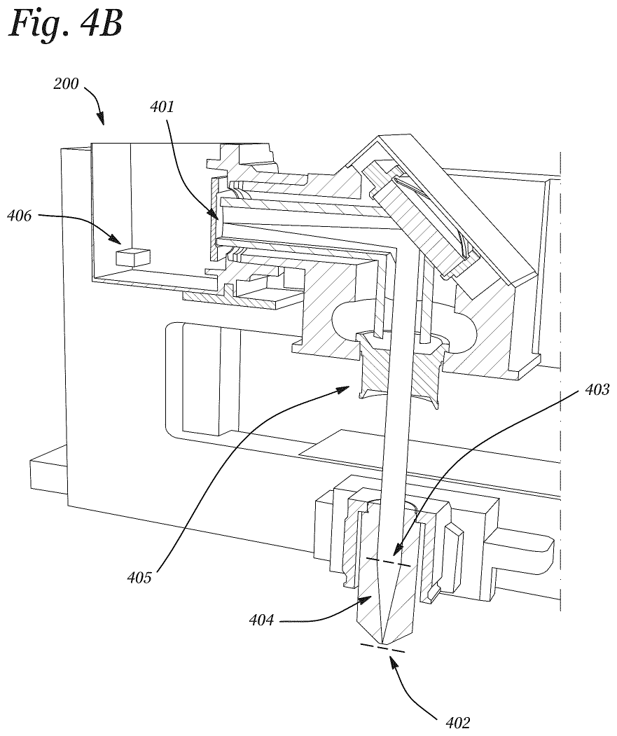

Referring now to FIG. 5, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random-access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments described herein.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to microcode, device drivers, redundant processing units, external disk drive arrays, RAID systems, tape drives, and data archival storage systems, etc.

The present disclosure includes systems, methods, and/or computer program products. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

### An Autofocus Module

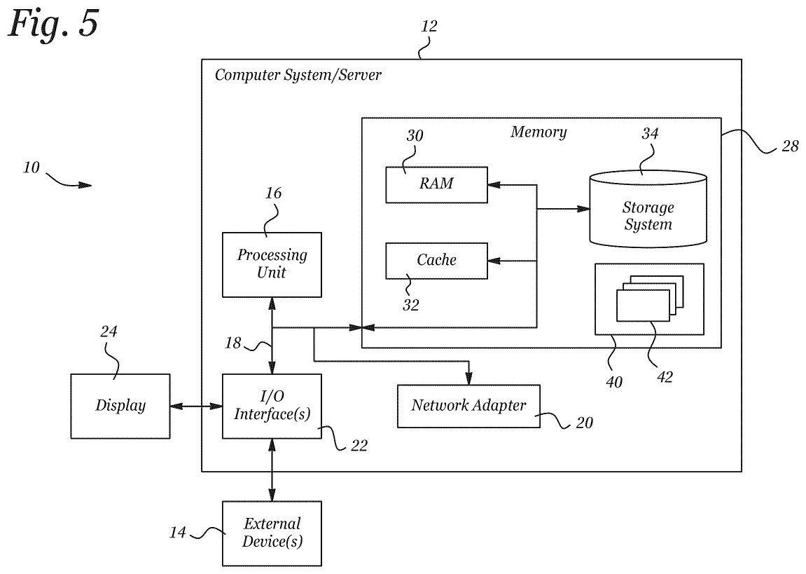

FIG. 6 illustrates an imaging system 500 (or an optics module thereof, such as described with respect to FIGS. 4A-4B) for fluorescence microscopy that includes an autofocus module 550. The imaging system 500 can be as described below and optionally can be based on (and include any or all features of) the optics module and/or the imaging system and/or the computer node described with reference to FIGS. 1-5. As shown in FIG. 6, the imaging system 500 includes an objective 502, a tube lens 504, and an image sensor 506. In some examples, the image sensor 506 is a camera, CCD or CMOS image sensor. In this setup, the objective 502 is an infinity corrected objective arranged collect emission light from a surface 508 positioned at or near to a focal plane of the objective. The surface 508 described is any reflective interface. The reflective interface may be a surface of a sample, a sample mount or the interface therebetween. The objective 502 is arranged to collimate the emission light. The collimated emission light passes from the objective 502 to the tube lens 504 through the infinity space 510. In implementations, the surface 508 is positionable at the focal plane of the objective 502. In particular embodiments, the sample surface is movable through the focal plane of the objective, particularly in the z-direction. In embodiments, the sample is carried on or supported by a mount, wherein the mount is movable through the focal plane. The sample is moveable, for example, in response to commands from a controller of the imaging system.

In the imaging system 500 of FIG. 6, illumination light 512 (also referred to as excitation light) is introduced from an illumination light source 511. The illumination light source 511 is configured to generate the illumination light 512. In some implementations, the illumination light source 511 is included as part of the imaging system 500 (e.g., as a module thereof). Otherwise, the illumination light source is external to the imaging system 500. In some implementations, such as depicted in FIG. 6 (e.g., in a Koehler illumination configuration), the imaging system includes a condenser (or field) lens 518 to form an image of the light source (e.g., the LED) at the back focal plane of the objective 502. As a result, the collimator provides collimated illumination light after the objective 502 and thus a uniform illumination profile at the sample surface 508.

The illumination light is selected such that its output spectrum corresponds to the excitation wavelength of a fluorophore (e.g., a red, yellow, green, or blue dye) in a sample on the surface 508. For instance, in some embodiments, at least a portion of the illumination light includes at least one fluorophore-dependent excitation wavelength for exciting a fluorophore in the sample. In response to being excited by the fluorophore-dependent excitation wavelengths, the fluorophores emit emission light, which the imaging system 500 is arranged to capture and image at the image sensor 506.

A dichroic element 514 is positioned in the infinity space 510 to intersect the illumination light 512 from the illumination light source 511. In many implementations the dichroic element 514 is a dichroic mirror. As such, the dichroic element 514 is also referred to herein as a first dichroic mirror or an illumination dichroic mirror. The dichroic element 514 has wavelength dependent reflectivity such that the dichroic element 514 is configured to reflect the illumination light 512 (e.g., to reflect a first spectral range that corresponds to the illumination light 512) and to transmit emission light (e.g., to transmit a second spectral range that corresponds to the emission light). In some embodiments, the first spectral range includes a (e.g., peak) wavelength corresponding to an excitation wavelength of a fluorophore in the sample on the surface 508. Similarly, in some embodiments, the second spectral range includes a (e.g., peak) wavelength corresponding to an emission wavelength of a fluorophore in the sample on the surface 508. Accordingly, positioning the illumination dichroic element 514 to intersect the illumination light 512, as shown in FIG. 6, provides that the illumination light is directed to the objective 502 by reflection from the dichroic element 514, and emission light collected by the objective 502 is directed to the tube lens 504 by transmission through the dichroic element 514. Accordingly, the illumination light source 511 is positioned to direct at least a portion of the illumination light 512 from the illumination light source 511 to the illumination dichroic element 514. In turn, the illumination dichroic element 514 is positioned to direct, by reflection, at least a portion of the illumination from the illumination light source 511 to the objective 502.

However, in other embodiments, the dichroic element 514 may be configured to direct the illumination light to the objective 502 by transmission therethrough, and direct emission light collected by the objective 502 to the tube lens 504 by reflection from the dichroic element 514. As such, in these other embodiments, the dichroic element 514 is configured to reflect the emission light and to transmit the illumination light. In these other embodiments, the positioning of components in the imaging system 500 is configured differently to as shown in FIG. 6. As an example, in these other embodiments the positions of the image sensor 506 and the illumination light source 511 are swapped.

The system of FIG. 6 finds use in imaging a sample (disposed on a surface 508) having a target analyte tagged with a fluorophore. In operation, illumination light 512 is directed from the illumination light source 511 to the illumination dichroic mirror 514. At the illumination dichroic mirror 514, the illumination light 512 is directed (reflected or transmitted) to the objective 502. In some embodiments, the objective 502 focuses the illumination light 512 onto the surface 508 or a sample thereon. In some embodiments, in a Koehler illumination configuration, the objective 502 delivers a uniform illumination profile to the sample surface 508. At least a portion of the illumination light 512 includes at least one of the fluorophore-dependent excitation wavelengths for exciting the fluorophore in the sample. As such, the fluorophore is excited by the illumination light 512. In response, the fluorophores emit emission light, which is directed through the objective 502 and the infinity space 510, via the dichroic plate 514 to the tube lens 504, and focused by the tube lens 504 onto the image sensor 506. The image sensor 506 captures the focused emission light so that an image of the fluorophores in the sample can be captured. In some embodiments, such as the embodiment depicted in FIG. 6, the emission light is passed through an optical filter 516 (e.g., a long pass filter, a short pass filter, a band pass filter) to filter out one or more wavelengths of light before arriving at the image sensor 506. In some embodiments, the optical filter 516 is optional and may be omitted.

In some embodiments, a z-distance between the objective 502 and the sample on the surface 508 is adjusted one or more times to thereby image parts of the sample at additional planes through the sample (also referred to as z-slices) within a field of view (FOV) of the objective 502. In some embodiments, a plurality of z-slices of a FOV form a z-stack of images representing the position of fluorophores in a volume of the sample. The z-distance (of offset) between the objective 502 and the sample may be adjusted by adjusting the position of the surface 508 in the z-direction. The z-direction is the direction perpendicular to the plane of the objective 502 and the surface of the sample, and/or z-direction is the direction along the optical axis of the objective 502.

The imaging system 500 includes autofocus module 550, which can detect the position of the surface 508 with respect to the objective to aid in capturing each z-slice at a precise plane within (or on the surface of) the sample. The autofocus module of the imaging system shown in FIG. 6 includes an autofocus light source (also referred to as a reference light source) 552, an additional dichroic mirror 554 (also referred to as a reference dichroic mirror 554), and an additional image sensor 556 (also referred to as a reference image sensor 556).

The autofocus light source 552 is arranged to generate a beam of reference light or a reference light spectrum (also referred to as autofocus light). In implementations, the reference light is selected such that it does not interfere with or otherwise affect the imaging of the sample. For instance, the autofocus light source 552 is selected such that the reference light does not excite a fluorophore in the sample. In examples, this is achieved by a peak wavelength of the reference light being orthogonal to (e.g., distant from) the excitation wavelength of the fluorophore. Some fluorophores have excitation wavelengths of up to around 700 nm. In some embodiments, the reference light has a peak wavelength in at least the red or infrared range. However, some fluorophores have lower excitation wavelengths, and therefore the reference light may include correspondingly lower wavelengths to match. For instance, the peak wavelength of the reference light may take any value above about 600 nm, for example above about 600 nm, above about 625 nm, above about 650 nm, above about 675 nm, above about 700 nm, above about 725 nm, above about 750 nm, above about 775 nm, above about 800 nm, above about 825 nm, above about 850 nm, above about 875 nm, above about 900 nm, above about 950 nm, or above about 1000 nm. In some examples, the reference light may have a peak wavelength in the infrared spectrum or lower. For instance, the peak wavelength may take any value below 1 mm, for example below 3 μm, or below 1.4 μm.

Preferably, the reference light is substantially spatially coherent (i.e., has a high degree of spatial coherence). This can reduce the degree of interference (e.g., diffraction patterns) introduced by the imaging system 500. On the other hand, interference such as diffraction patterns introduced in the imaging system can be modelled and/or filtered out of the reflected reference light and therefore the reference light need not be spatially coherent. In some embodiments, the reference light is additionally or alternatively substantially temporally coherent (i.e., has a high degree of temporal coherence). However, in certain implementations, the light need not be temporally coherent. The autofocus light source may comprise a laser, an LED source, or a superluminescent diode. As will be understood, lasers are usually temporally and spatially coherent, many LED sources are neither temporally nor spatially coherent, and many superluminescent diodes are spatially, but not temporally, coherent.

The reference dichroic mirror 554 has wavelength dependent reflectivity. The reference dichroic mirror 554 of the imaging system 500 of FIG. 6 is configured to reflect the reference light and transmit both the illumination light 512 and the emission light. In addition, the reference dichroic mirror 554 is positioned in the infinity space. As such, the reference dichroic mirror 554 is positioned to direct at least a portion of the reference light from the autofocus light source 552 to the objective 502 and to direct at least a portion of the reference light reflection captured by the objective 502 to the reference image sensor 556. In some examples, the reference image sensor 556 is a camera, CCD, CMOS image sensor, or any other image sensor suitable for capturing and/or sensing the reflection of the reference light.

The autofocus module 500 is arranged to use a triangulation method to track the position of the surface 508 relative to the objective. The triangulation method is now described with reference to the autofocus module 500 in FIG. 6. The autofocus light source 552 and the reference dichroic mirror 554 are respectively disposed such that the reference light, when reflected off the reference dichroic mirror 554, is directed to an off-axis position of the objective 502. As such, the reference light beam is refracted by the objective 502. After passing through the objective 502, the reference light beam is incident on and reflects off the surface 508 at an oblique angle to the objective optical axis. The reflection of reference light (i.e., the reflected beam) is then collected by the objective 502. As the reference light beam is incident on the surface at an oblique angle, the position of incidence on the surface 508 varies with the distance between the objective 502 and the surface 508 in the z-direction. The position of incidence on the surface affects the position on the objective 502 at which the reflection of reference light is collected by the objective 502. In turn, this affects the position of the reflection in the infinity space relative to the objective optical axis and therefore the position of the reflection on the reference image sensor 556. As such, in the autofocus module 550 of the imaging system 500 of FIG. 6, the (e.g., centroid) position at which the reflection of reference light off the surface 508 is incident on the reference image sensor 556 is dependent on the distance between the objective 502 and the surface 508 in the z-direction.

The imaging system 500 is arranged to identify a relationship between the position of the reflected reference beam on the image sensor and a position of the surface 508 (or reflective interface) in the field of view of the objective from which the reference beam is reflected. The imaging system is arranged to identify a difference between the reflection of the reference light incidence position on the reference image sensor 556, and a reference position on the image sensor. For instance, in some embodiments the reference position corresponds to a known position of the surface 508 relative to the objective 502, for example known position may be the focal plane of the objective. In examples, the known position corresponds to the position of a plane relative to a sample. Accordingly, from the difference between the detected and reference positions of the reflected reference beam on the image sensor, the distance between the focal plane of the objective 502 and the surface 508 can be determined. The autofocus module can therefore verify where the sample (or each respective z-slice of the sample) is positioned relative to the focal plane of the objective 502. In some examples, a plurality of reference positions on the image sensor may be considered, each of which correspond with a z-direction position relative to the focal plane of the objective. A respective image can be obtained by the emission image sensor (the image sensor detecting the emission light) at each of these z-direction positions to make up the z-stack of images representing an image volume. In some embodiments, the z-distance between the objective 502 and the sample on the surface 508 is adjusted one or more times to thereby image additional planes in the sample using the emission image sensor. As described herein, in some embodiments, a plurality of z-slices of a FOV form a z-stack of images to build up an image of the fluorophores throughout the volume of the sample.

While optical components (e.g., components corresponding to peripheral functions of the microscopes such as the autofocus module 550) can be positioned within the infinity space, the amount of available infinity space is limited. In some embodiments, the infinity space is about 50 mm to about 200 mm long, measured between the objective 502 and the tube lens 504. In some embodiments, the infinity space is about 100 mm to about 200 mm measured between the objective 502 and the tube lens 504. In some embodiments, the infinity space is about 120 mm to about 160 mm measured between the objective 502 and the tube lens 504. In some embodiments, the infinity space is about 100 mm measured between the objective 502 and the tube lens 504. In other embodiments, the infinity space may take any suitable length. The reference dichroic mirror 554 takes up a portion of the infinity space. A problem therefore exists in how to increase availability of infinity space for other peripheral functions, while retaining the functionality of the autofocus module. Another problem is how to reduce the infinity space to reduce the overall volume or footprint of the imaging system, while retaining the functionality of the autofocus module.

Furthermore, optical components heat up under irradiation from high intensity light, such as the illumination light 512. Optical components can also absorb heat from the environment. The operational behaviour of the optical components can change over time as result of thermal drift, for example during prolonged use of the imaging system. These operational behaviour changes can be changes in focal length and alignment, which reduces the performance of the imaging system 500 during prolonged use. A problem therefore exists in how to increase the accuracy and reliability of the autofocus module during prolonged use.

### Improved Imaging System

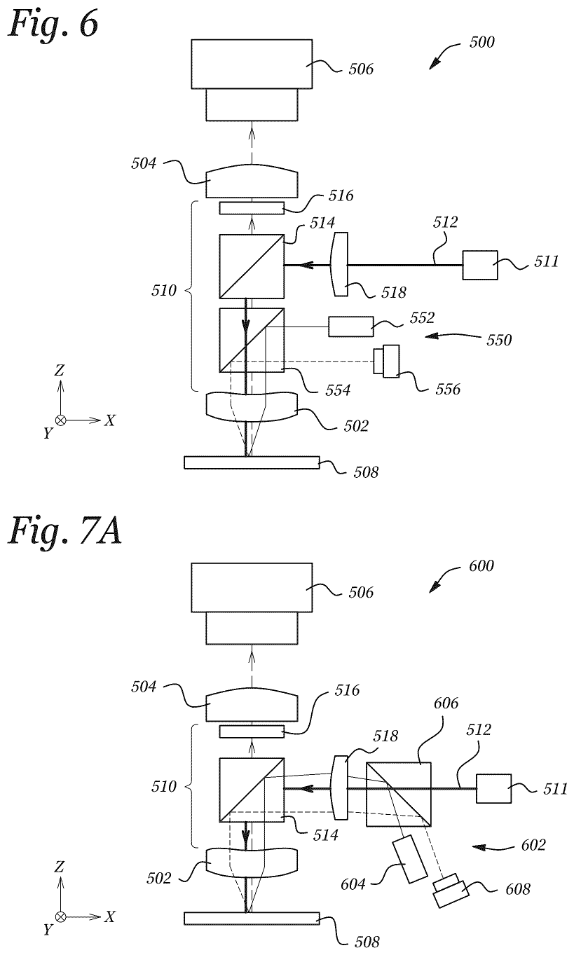

FIGS. 7A-7C illustrate embodiments of an imaging system 600, 630, 660 for fluorescence microscopy according to embodiments of the present disclosure. The imaging systems of FIGS. 7A-7C can operate as part of the imaging systems described with reference to FIGS. 4A and 4B and/or can include the computing node described with reference to FIG. 5. The imaging systems 600, 630, 660 of FIGS. 7A-7C include many of the same features as the imaging system 500 of FIG. 6. In implementations, and unless otherwise stated, the features and layouts of FIGS. 7A-7C are the same as the corresponding features and layouts described with respect to the imaging system 500 of FIG. 6. For instance, the embodiments of the imaging system 600, 630, 660 depicted in FIGS. 7A-7C include an objective 502 and a tube lens 504, between which is an infinity space 510 in which an illumination dichroic mirror 514 is disposed. An image sensor 506 is positioned at the focal plane of the tube lens 504. Accordingly, illumination light 512 is introduced, optionally via a condenser lens 518, to the infinity space 510. The illumination light 512 is transmitted by the illumination dichroic mirror 514 to the objective 502, where the illumination light is delivered to a focal plane at or about a sample on a surface 508. The illumination light 512 excites fluorophores in the sample. As a result, emission light is emitted by the fluorophores. The emission light is collected by the objective 502, transmitted through the illumination dichroic mirror 514, optionally via optical filter 516, to the tube lens 504 where it is focused onto the image sensor 506.

In embodiments, the autofocus system 602, 632, 662 is arranged to increase infinity space 510 availability in the imaging system 600, 630, 660 or otherwise to allow the length of the infinity space to be shortened. Infinity space 510 availability is increased, for instance, by minimising the number of components of the autofocus system 602, 632, 662 disposed in the infinity space 510. Minimising the number of components of the autofocus system can instead (or in addition) allow the length of the infinity space to be shortened. In many examples, the autofocus system 602, 632, 662 includes no components in the infinity space 510. The inventors have recognised these and other benefits of maximising the number of components of the autofocus system outside of the infinity space, or even providing all of the components of the autofocus system outside of the infinity space. Unless described herein, the infinity space includes the space directly between the objective and the tube lens.

In some embodiments, the autofocus system 602, 632, 662 is arranged to provide improved accuracy during prolonged use, for example by minimising the difference in thermal drift between the reference beam on the one hand, and the illumination and emission beams on the other. This may be achieved by extending the length of the shared optical path of (i) the reference beam reflection between the sample (or reflective interface) and the reference image sensor; and (ii) the emission light between the sample and the emission image sensor. In some implementations the reference image sensor and emission image sensor are the same component. In some embodiments, this is achieved by selecting the illumination dichroic so that it directs the reference light reflected from the surface (or reflective interface) in the same way as it directs the emission light from the sample. For example, the illumination dichroic is arranged to allow both the emission light and the reflected reference light to be transmitted therethrough or to be reflected. In this case, the imaging system can employ the same image sensor to detect (or capture, or sense, or image) both the reflected reference light and the emission light. For instance, in the autofocus system 602, 632, 662 of FIGS. 7A-7C the reference light and the illumination light 512 share an optical path between the condenser (or field) lens 518 and the sample 508. In the autofocus system of FIGS. 7B and 7C the emission light and the reflected reference light share an optical path between the sample 508 and the image sensor 506. As such, any operational behaviour changes—due to, for instance, thermal drift—in the shared optical path affects the reference light in the same way as the illumination light, and/or affects reflected reference light in the same way as the emission light. As such, the autofocus system can account for changes in optical properties, for instance, the objective 502 focal distance or illumination dichroic mirror 514 alignment.

The illustrated embodiments of the autofocus system 602, 632, 662 include an autofocus light source 604 configured to generate reference light. The autofocus light source 604 is as described with respect to the autofocus light source 552 of FIG. 6. The reference light source is selected such that output spectrum of the reference light does not interfere with or otherwise affect the sample on the surface 506. In some embodiments and as discussed herein with respect to FIG. 6, the autofocus light source 552 is selected such that the reference light does not excite a fluorophore in the sample. Preferably, the reference light is spatially coherent (i.e., has a high degree of spatial coherence). As an example, the reference light may comprise a laser, an LED projector, or a superluminescent diode.

The autofocus system 600, 630, 660 is configured to increase infinity space availability and increase autofocus accuracy during prolonged use. The autofocus system achieves one or more of these effects by directing the reference light from the autofocus light source 604 to the illumination dichroic mirror 514 such that the optical axis of the reference light is incident on the illumination dichroic mirror 514 parallel to the optical axis of the illumination light. As such, the autofocus system 600, 630, 660 is configured to introduce the reference light into the infinity space 510 parallel with the optical axis of the illumination light 512. Here, the illumination dichroic mirror 514 is additionally arranged to direct at least a portion of the reference light to the objective. Accordingly, in examples, the wavelength dependent reflectivity of the illumination dichroic mirror 514 is such that the illumination dichroic mirror 514 is configured to reflect the reference light (e.g., a third spectral range that corresponds to the reference light) as well as reflect the illumination light.

As such, the autofocus light source 604 is positioned and the reference light directed such that the optical path of reference light from the autofocus light source 604 intersects the illumination dichroic mirror 514. In embodiments, such as those shown in FIGS. 7A-7C, the optical path of reference light intersects the illumination dichroic mirror 514 in a parallel direction to the optical path of the illumination light 512. In these or other embodiments, the autofocus light source 604 is positioned such that the beam of reference light overlaps with at least a portion of the beam of illumination light 512. In some embodiments, the beam of reference light is narrower than the beam of illumination light 512 and is entirely contained within the beam of illumination light 512. In these embodiments, when the optical axis of illumination light 512 at the objective 502 is concentric with the optical axis of the objective 502, the optical axis of the reference light is offset from the optical axis of the illumination light 512. The optical axis of the reference light at the objective 502 is offset from the optical axis of the objective 502. In many implementations, the optical axes of the reference light and the illumination light 512 are parallel or substantially parallel to the optical axis of the objective 502 on incidence with the objective 502.

In some implementations, the reference light is active (i.e., switched on) when the illumination light is inactive (i.e., switched off), and vice versa. Therefore, in some implementations—for instance when the autofocus light source 604 is positioned such that the beam of reference light overlaps with at least a portion of the beam of illumination light 512—the reference light and the illumination light 512 are not active concurrently. The reference light and illumination light may be provided alternately. The reference light may be used between generating image frames using the illumination light.

As shown in FIGS. 7A and 7B, embodiments of the imaging system 600, 630, are arranged to increase the length of optical path shared between the reference light and the illumination light 512 by introducing the reference light into the infinity space from the same direction as the illumination light 512. For instance, the autofocus light source 604 is positioned to introduce reference light into the infinity space from the same direction as the illumination light 512.

In some implementations, the imaging system 600, 630 may further comprise an illumination module (which may include the illumination sources 210 described herein with respect to FIGS. 4A-4B, also referred to herein as (illumination) light sources). The illumination module comprises the (illumination) light source configured to generate illumination light and direct the illumination light to the illumination dichroic mirror. In some implementations, the illumination module additionally includes the condenser lens 518 (if present) shown in FIGS. 7A-7C. Otherwise, in some implementations, the condenser lens 518 is external to the illumination module. When present, the illumination module may additionally comprise the autofocus system 603 or any component thereof. For instance, the autofocus light source 604 may be provided in (or as part of) the illumination module. By providing an illumination module comprising the light source, the light source of the imaging system 600, 630, 660 can be interchangeable and/or discretely configurable according to the desired use and/or analyte in the sample. Providing at least one component of the autofocus system in the illumination module increases the length of optical path shared between the reference light and the illumination light 512, which increases the accuracy of the autofocus system during prolonged use because thermal drift affects the optical path of the reference light substantially equally to the optical path of the illumination light 512.

In some implementations, the illumination module is configured to selectively generate illumination light of different wavelengths, such as different wavelengths corresponding to different fluorophores in the sample. By doing so, one illumination module can excite multiple analytes and/or fluorophores. In examples, the illumination module includes a plurality of light sources. The illumination module is independently controllable to present one of the light sources to the imaging system. In other words, the illumination module is independently controllable to change the wavelength of the illumination light 512 by selecting one of the light sources as the source of the illumination light 512. In some of these examples, the illumination module is arranged to change the light source (i.e., change the wavelength of the illumination light 512) presented to the imaging system by moving the plurality of light sources relative to the imaging system so that a different light source introduces illumination light into the imaging system at any one time. In this way, the illuminator module can scroll through each light source, i.e., by moving one illumination source at a time into the required position to introduce illumination light into the imaging system. In some of these embodiments, the autofocus system 602, 632 (or at least a component thereof, such as the autofocus light source 604) is arranged to remain stationary during movement of the illumination light sources to provide consistent reference light for focusing the system, and thereby increasing focusing accuracy over all illumination light sources of the illuminator module.

In some implementations, such as those shown in FIGS. 7A-7C, the imaging system 600 includes a condenser (or field) lens 518. As described herein, the condenser lens 518 is present in an illumination module or is provided outside an illumination module. The condenser lens 518 is arranged to collect the illumination light 512 from the light source and transmit the illumination light 512 to the illumination dichroic mirror 514. In some embodiments, the condenser lens 518 has positive optical power. In some implementations, the condenser lens 518 is a refractive optic, such as a lens.

In some embodiments, such as shown in FIGS. 7A and 7B, the autofocus system is arranged to direct the reference light to incident the condenser lens 518 at an oblique angle. The condenser lens 518 is arranged to direct the obliquely incident reference light to a direction parallel to the optical axis. As a result, the condenser lens 518 transmits the beam of illumination light in parallel to the beam of reference light between the condenser lens 518 and the illumination dichroic 514.

In some embodiments, such as shown in FIGS. 7A and 7B, the autofocus system 602, 632 further comprises an additional dichroic mirror 606 (also referred to as a reference dichroic mirror 606). In many implementations, the reference dichroic mirror 606 is as described with respect to reference dichroic mirror 554 of the imaging system 500 of FIG. 6, except in that the reference dichroic mirror is not positioned in the infinity space. For instance, the reference dichroic mirror 606 has wavelength dependent reflectivity such that it is configured to reflect (at least a portion of) the reference light from the autofocus light source 604. The reference dichroic mirror 606 is configured to transmit (at least a portion of) the illumination light 512. In the embodiments of the imaging system 600, 630 depicted in FIGS. 7A and 7B, the reference dichroic mirror 606 is not present in the emission light path.

The reference dichroic mirror 606 is configured to direct the reference light from the autofocus light source so that the reference light beam is introduced into the infinity space parallel to the beam of illumination light (i.e. the beams have parallel optical axes). The reference dichroic mirror 606 is configured and positioned to direct the reference light from the autofocus light source 604 to the illumination dichroic mirror 514, via the condenser lens 518 (if present). For instance, the reference dichroic mirror 606 is configured to reflect the reference light beam (or at least a portion thereof) from the autofocus light source 604 to the condenser lens 518, at an oblique angle to the optical axis of the condenser lens 518. Alternatively, for instance if the condenser lens 518 is not present, the reference dichroic mirror 606 is configured to reflect at least a portion of the reference light from the autofocus light source 604 to the illumination dichroic mirror 514, in a direction parallel to the optical axis of the illumination light.

Accordingly, in embodiments of the imaging system 600, 630 shown in FIGS. 7A and 7B, reference light is introduced by the autofocus light source 604 and directed to the illumination dichroic mirror 514. The reference light is directed to the illumination dichroic mirror by reflection from the autofocus dichroic mirror 606 (if the autofocus dichroic mirror 606 is present) and towards the condenser lens 518 (if the condenser lens 518 is present), at an oblique angle to the optical axis of the condenser lens 518. The reference light then passes to the illumination dichroic mirror 514, parallel to the optical axis of the illumination light 215, where the reference light is reflected by the illumination dichroic mirror 514 in the infinity space 510, towards the objective 502.

Embodiments of the imaging system 660 shown in FIG. 7C differ from the embodiment of the imaging system 600, 630 shown in FIGS. 7A and 7B in that the autofocus system 662 of FIG. 7C is not provided within or as part of the illumination module. Instead, the light source 604 of the autofocus system 662 is disposed and arranged to introduce reference light into the imaging system 660 (or into the infinity space) in a different direction to the illumination light. In these embodiments, the light source 604 is arranged to direct the reference light towards the autofocus dichroic mirror 606 such that a reflection of the reference light from the autofocus dichroic mirror 606 is parallel to the optical axis of the illumination light, at least at the point at which the illumination light 512 is incident on the illumination dichroic mirror 514. In some of these embodiments, the autofocus dichroic mirror 606 is provided downstream of the condenser lens 518 (if present), as shown. In many embodiments, the autofocus dichroic mirror 606 is configured in any of the manners discussed herein, particularly with respect to FIGS. 7A and 7B. As shown, the autofocus dichroic mirror 606 is provided outside the infinity space 510 and is configured such that the reference light passes along the same optical path through the illumination dichroic mirror 514 and the objective 502 as the illumination light 512. Accordingly, the autofocus system 662 of FIG. 7C can provide the same improvements to infinity space availability and autofocus accuracy during prolonged use discussed herein with respect to FIGS. 7A and 7B.

In the embodiment of the imaging system 660 shown in FIG. 7C, reference light is introduced by the autofocus light source 604. The reference light is reflected off the autofocus dichroic mirror 606 and towards the illumination dichroic mirror 514, at which the reference light is reflected, in the infinity space 510, towards the objective 502. As shown, the autofocus light source 604 and autofocus dichroic mirror 606 are positioned to reflect the reference light parallel to the optical axis of the illumination light 512 and, when reflected from the illumination dichroic mirror, offset from the optical axis of the objective 502. Thus, the optical axes of the reference light and illumination light 512 are parallel to each other in the infinity space between the illumination dichroic 514 and the objective 502.

In the embodiments of the imaging system 600, 630, 660 shown in FIGS. 7A-7C, the reference light passes to the objective 502 after reflection from the illumination dichroic mirror 514. As discussed in more detail herein, in many implementations, the optical axis of the reference light is incident on the objective 502 at a parallel (or substantially parallel) and offset position relative to the optical axis of the objective 502. As the reference light is offset relative to the optical axis of the objective 502, the reference light is refracted by the objective 502. The refracted reference light is then incident on the surface 508 at an oblique angle, at which it is reflected, and the reflection is collected by the objective 502. As described herein, particularly with respect to FIG. 6, the position of incidence on the surface 508 varies with the distance between the objective 502 and the surface 508 in the z-direction. The position of incidence on the surface affects the position on the objective 502 at which the reflection of reference light is collected by the objective 502. Once collected, the reflection of the reference light passes through the pupil of the objective 502 and to the infinity space 510. From the infinity space, the reflection of the reference light is received at an image sensor 506, 608 in the manner described herein.

Embodiments of the imaging system 600, 630, 660 shown in FIGS. 7A-7C further include an image sensor 506, 608. The image sensor 506, 608 is configured to receive a reflection of the reference light from the surface 508. Further, as discussed herein with respect to FIG. 6, by comparing the position of the reflection of the reference light on the image sensor to a reference position on the image sensor, a z-distance between the objective 502 and a sample on the surface 508 can be determined.

In some embodiments, the imaging systems described herein further comprise computer hardware, such as the computing node 10 described with reference to FIG. 5. In some implementations, the computer hardware may comprise one or more processors that are configured to identify a detected position of the reflection of the reference light on the image sensor. Using the detected position, the one or more processors are configured to determine a position of the surface 508 relative to the objective 502 or relative to a focal plane of the objective.

In some implementations, the one or more processors are arranged to identify a relationship (e.g., a difference) between the detected position of the reflected reference beam on the image sensor and a reference position on the image sensor 506, 608. For instance, the one or more processors are in communication with the reference image sensor 506, 608 such that the detected position and/or the Euclidian distance from the detected position to the reference position can be communicated from the image sensor 506, 608 to the one or more processors. A position of the surface 508 relative to the objective 502 or relative to a focal plane of the objective is determined by the one or more processors from the detected position or the distance between the reference position and the detected position on the image sensor. In the imaging system shown in FIG. 7A, the image sensor comprises a reference image sensor 608 of the autofocus system 602. The reference image sensor 608 is disposed to receive the reflection of the reference light from the surface 508, via the objective 502 and the illumination dichroic mirror 514. Unless otherwise described the reference image sensor 608 of FIG. 7A is as described with respect to the reference image sensor 556 of FIG. 6. In embodiments, the reference image sensor 608 is a camera, CCD, CMOS image sensor, or any other image sensor suitable for capturing the reflection of the reference light. By providing a reference image sensor 608 in addition to the image sensor 506, modularity of the autofocus system 602 can be retained, which can reduce complexity of installation and/or maintenance or repair of the imaging system.

In the imaging system shown in FIG. 7A, the reflection of the reference light is received from the infinity space at the image sensor 608 via reflection from the illumination dichroic mirror 514. In some implementations, the reference light reflection is received through the condenser lens 518 (if present) and by reflection off the reference dichroic mirror 606, as shown in FIG. 7A. Since the distance between the objective 502 and the surface 508 in the z-direction affects the position on the objective 502 at which the reflection of reference light is collected, the distance between the objective 502 and the surface 508 in the z-direction also affects the lateral position of the reference light reflection in the infinity space, which in turn affects the position of the reflection when reflected by the illumination dichroic mirror 514, transmitted through the condenser lens 518, and reflected by the reference dichroic mirror 606 to the image sensor 608. Accordingly, the position of the reflection of the reference light on the image sensor 608 varies with the distance between the objective 502 and the surface 508 in the z-direction.

In the imaging systems described herein, for example those described with reference to FIGS. 7A-7C, by identifying the (e.g., centroid) position of the reflected reference light on the image sensor, a position of the surface 508 relative to the objective or the objective focal plane can be derived. For example, from a relationship (e.g., a difference) between the detected position of the reflection of the reference light on the reference image sensor 608, and a reference position on the image sensor 608 corresponding to a known distance, the distance between the focal plane of the objective 502 and the surface 508 can be determined. In embodiments, the reference position corresponds to a reference plane. The reference plane is a plane in the field of view of the objective arranged perpendicular to the optical axis of the objective. The reference plane may be the objective focal plane or a plane that has a known separation distance from the objective or the objective focal plane. A collocation of the detected position and the reference position corresponds to a collocation of the surface 508 and the reference plane. In other words, if the detected position matches the reference position, the surface is at the reference plane. Triangulation techniques known to the skilled person, in conjunction with measurements between the optical components of the imaging system, can be used to determine the relationship between the position of the surface 508 and the detected (e.g., centroid) position of the reflected reference light on the image sensor.

In, for example, the imaging systems 630, 660 shown in FIGS. 7B and 7C, an emission-receiving image sensor 506 is configured to receive a reflection of the reference light from the surface 508. In such implementations, the image sensor 506 is configured to and suitable for receiving and detecting both: emission light from the sample on the surface 508; and the reflection of the reference light. In these embodiments the image sensor 506 may be any image sensor suitable for capturing images of both emission light and the reflection of the reference light from the surface 508. For instance, as described with respect to FIG. 6, in some embodiments the image sensor 506 is a camera, CCD or CMOS image sensor.

In the imaging systems depicted in FIGS. 7B and 7C, the illumination dichroic mirror 514 is configured to transmit both the emission light (as in the embodiments shown in FIGS. 6 and 7A) and the reflection of the reference light. To achieve this function, the illumination dichroic mirror may be partially reflective. For instance, the illumination dichroic mirror 514 is configured to both reflect the reference light (e.g., a first spectral range that corresponds thereto) and to transmit the reflection of the reference light (which has, for instance, the same first spectral range). In these embodiments, the illumination dichroic mirror 514 may have substantially 50% reflectivity (e.g., 50% reflectivity; higher than 20%, 30%, 40%, or 45% reflectivity; and/or lower than 80%, 70%, 60%, 55% reflectivity) to the spectral range of the reference light. Alternatively, or in addition, some portions of the dichroic surface of the illumination dichroic mirror may be arranged to have a higher reflectance than other portions to allow a high proportion (e.g., greater than 50%) of the reference beam to be delivered to the surface 508 while also allowing a high proportion (e.g., greater than 50%) of the reflected reference beam to be transmitted therethrough to be captured by the image sensor 506. For example, in the illumination dichroic mirrors shown in FIGS. 7B and 7C, the (reflective) dichroic surface to the right of the optical axis of the objective and tube lens has a first reflection co-efficient for the reference light and the dichroic surface to the left of the optical axis of the objective and tube lens has a second reflection co-efficient for the reflected reference light. In such implementations, the first reflection co-efficient is higher than the second reflection co-efficient.

In imaging systems of the present disclosure, for example those described with reference to FIG. 7C, the tube lens 504 is further configured and positioned to focus the reflection of the reference light onto the image sensor 506 at the imaging plane thereof. Here, when the optical filter 516 is provided, the filter is also selected to transmit light in the same spectral range as the reference light.

In the imaging systems of FIGS. 7B and 7C, the reflection of the reference light is received from the infinity space at the image sensor 506 via transmission through the illumination dichroic mirror 514 and the tube lens 504. The tube lens 504 focuses the reflection of the reference light to the imaging plane (e.g., at the image sensor 506). Since the distance between the objective 502 and the surface 508 in the z-direction affects the position on the objective 502 at which the reflection of the reference light is collected, the distance between the objective 502 and the surface 508 in the z-direction also affects the position of the reflection in the infinity space, which affects the position of the reflection on the focal plane of the tube lens 504. Accordingly, the position of the reflection of the reference light on the image sensor 506 varies with the distance between the objective 502 and the surface 508 in the z-direction. As such, by identifying a difference between the detected position of the reflection of the reference light on the image sensor 506, and a reference position on the image sensor 506 corresponding to a known distance, the distance between the focal plane of the objective 502 and the surface 508 can be determined. The detected position of the reflected reference beam can be used in any of the other ways described in the present disclosure to determine the position of the surface 508 relative to the objective or objective focal plane.

By using a single image sensor for both the reference light and the emission light, the imaging systems 630, 660 described herein can be manufactured at reduced cost. In addition, the optical path of emission light (i.e., the path taken by the emission light between the sample and the image sensor 506) has increased overlap with an optical path of the reference light reflection (i.e., the path taken by the reflected reference light between the surface 508 and the image sensor 506). Having a single image sensor for both the reference light and the emission light allows the reflection of reference light to travel through the same components as the emission light. This reduces the effect of thermal drift on the accuracy of the autofocus system. The accuracy of the autofocus system (e.g., during prolonged use) is increased because operational behaviour changes of optical components affect the emission light in substantially the same way as the reference light reflection.

It will be appreciated that many embodiments may combine features from the imaging systems 600, 630, 660 shown in each of FIGS. 7A-7C. For instance, in some alternative embodiments not shown in the figures, an additional reference image sensor 608, such as is shown in FIG. 7A, may be provided to the embodiment of the imaging system 660 shown in FIG. 7C. For instance, in these embodiments the reflection of the reference light is received from the infinity space at the image sensor 608 by reflection off the illumination dichroic mirror 514, and the reference dichroic mirror 606. As such, the reference image sensor 608 is positioned to receive the reflection of the reference light from the reference dichroic mirror 606. In these embodiments, the illumination dichroic mirror may be highly reflective to the reference light (such as by having a greater than 50%, greater than 60%, greater than 70%, greater than 80%, greater than 90% or substantially 100% reflectivity with respect to the spectral range of the reference light).

### Methods for Focusing an Imaging System

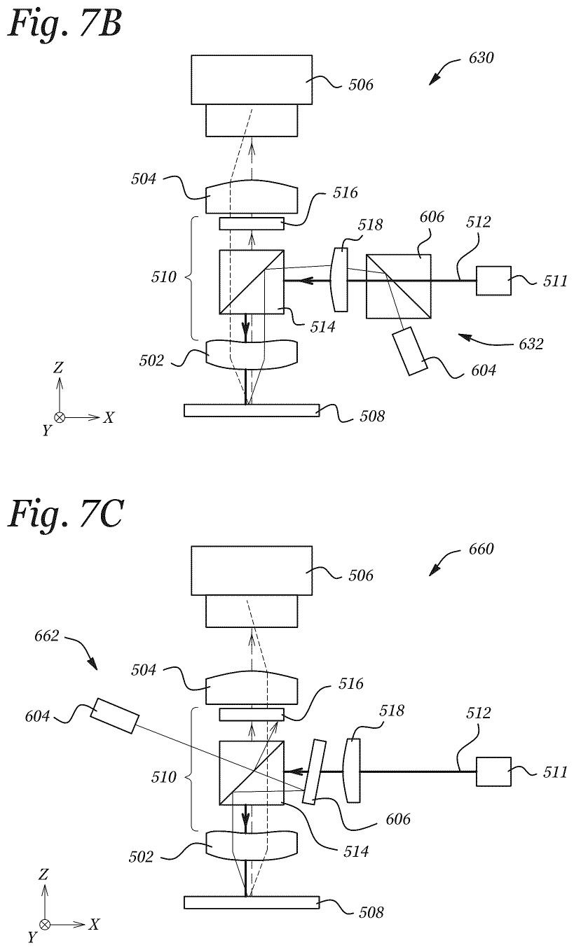

FIG. 8 shows an example method for focusing in an imaging system. The method of FIG. 8 is carried out with any of the imaging systems described herein, for example any of the imaging systems described with respect to FIGS. 7A-7C or any of their described variants. In some implementations, one or more steps of the method may be carried out by computer hardware, such as the computing node 10 described with reference to FIG. 5 or one or more processors. In some implementation, one or more steps of the method may be carried out manually.

The method begins at step S100 in which reference light is directed to an illumination dichroic mirror, such as illumination dichroic mirror 514 described herein. The reference light is generated by an autofocus light source of the imaging system, such as autofocus light source 552 or 604. In some implementations, directing the reference light to the illumination dichroic mirror comprises orienting the autofocus light source to direct reference light therefrom to the illumination dichroic mirror and activating the autofocus light source. In some implementations, the autofocus light source is oriented to direct reference light therefrom to the illumination dichroic mirror via a reference dichroic mirror 606 and a condenser lens 518, if present.

The method continues to step S105 at which a position of incidence of a reflection of the reference light from a surface on an image sensor is detected. In embodiments described herein, the surface is surface 508 and the image sensor is, for instance, any of image sensors 556, 608 or 506. As described with respect to the imaging systems of FIGS. 6 and 7A-7C, the reference light beam is incident on and reflects off the surface 508 at an oblique angle to the normal of the surface before being collected by the objective 502. As the reference light beam is incident on the surface at an oblique angle, the position of incidence on the surface 508 varies with the distance between the objective 502 and the surface 508 in the z-direction. Accordingly, the (e.g., centroid) position at which the reflection of the reference light off the surface 508 is detected on the image sensor is dependent on the distance between the objective 502 and the surface 508 in the z-direction.

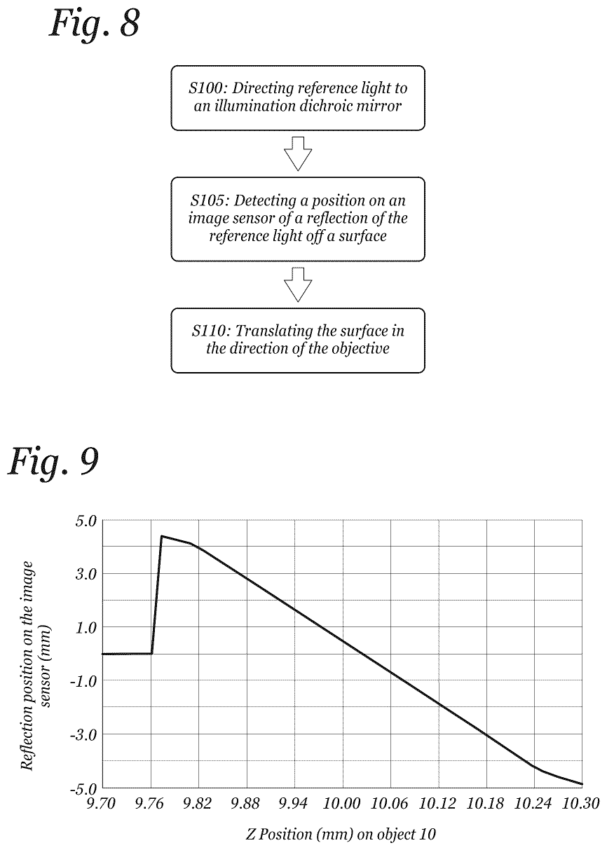

FIG. 9 shows a graph of the position of a surface (e.g., surface 508) relative to an objective (e.g., objective 502) on the horizontal axis vs. centroid position of a reflection of the reference light on an image sensor (e.g., any of image sensors 556, 608 or 506). As shown in FIG. 9, these two parameters are linearly correlated. As shown in FIG. 9, the linear correlation has a negative slope.

In some implementations, step S105 is carried out by computer hardware (or one or more processors) communicatively coupled to the image sensor. In some examples, the computer hardware is arranged to: identify the (e.g., centroid) position of the reflection of the reference light on the image sensor (e.g., on the pupil of the image sensor); and compare the position of the reflection on the image sensor to a reference position. In other examples, the computer hardware is arranged to output an indication of the position of the reflection on the image sensor to a display. In some of these examples, the display is arranged to display the position of the reflection, and optionally the reference position, to—for instance—a user, thus allowing a user to determine the relationship between the position of the reflection and the reference position.

The method continues to step S110 at which, the surface is translated relative to the objective in the z-direction, i.e., along the optical axis of the objective. The translation may be either towards or away from the objective. As shown in FIG. 9, translation in the z-direction linearly shifts the reflection position on the image sensor. In some implementations, step S110 involves translating the surface until the (e.g., centroid) position of the reflection is at a reference position on the image sensor, or within a (tolerance) threshold distance from the reference position. The reference position may correspond to a reference plane which is at a known or desired distance from the objective, for example at the objective focal plane or a known distance therefrom. In some examples, the reference position corresponds to a specific z-slice in the sample. In some implementations, the reference position is pre-calibrated.

In some implementations, translating the surface until the position of the reflection is at a reference position includes continuous detecting of the position of the reflected reference light on the image sensor, as in step S105. The continuous detection may be carried out by computer hardware or one or more processors. In one example, the computer hardware or one or more processors are arranged to detect the position of the reflection on the image sensor and, responsive to determining that the position of the reflection on the image sensor is outside a tolerance threshold distance from the reference position, actuating surface translation until the position of the reflection on the image sensor is within a tolerance threshold distance from the reference position. In another example, the computer hardware is arranged to detect a displayed distance of the reflection from the reference position, calculate the amount of surface translation that corresponds to the displayed distance, and actuate the surface translation by the calculated amount accordingly. In other implementations, the translation of the surface may be user controlled in response to viewing the display arranged to display a representation of the position of the reflection of the reference beam on the image sensor, and optionally the reference position.

### General Terminology

Specific terminology is used throughout this disclosure to explain various aspects of the methods, systems, and compositions that are described. Unless otherwise defined, all of the technical terms used herein have the same meaning as commonly understood by one of ordinary skill in the art in the field to which this disclosure belongs.

It is to be understood that certain terminology is used in the preceding description for convenience and is not limiting. The terms “a”, “an” and “the” should be read as meaning “at least one” unless otherwise specified. The term “comprising” will be understood to mean “including but not limited to” such that systems or method comprising a particular feature or step are not limited to only those features or steps listed but may also comprise features or steps not listed. Similarly, any features included as examples are not to be construed as limiting to the disclosure. Additionally, any combination of features from one implementation with features from one or more other example(s) is to be understood as within the present disclosure. Equally, terms such as “after”, “before”, “in front”, “behind”, “downstream”, “upstream” and so on are used for convenience in interpreting the drawings and are not necessarily to be construed as limiting in absolute terms. Additionally, any method steps which are depicted in the figures as carried out sequentially, without causal connection, may alternatively be carried out in series in any order. Further, any method steps which are depicted as dashed or dotted flowchart boxes are to be understood as being optional.

As used in this specification and the appended claims, the singular forms “a”, “an”, and “the” include plural references unless the context clearly dictates otherwise. For example, “a” or “an” means “at least one” or “one or more”. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements or method steps.

As used herein, the term “about” a number refers to that number plus or minus 10% of that number. The term ‘about’ when used in the context of a range refers to that range minus 10% of its lowest value and plus 10% of its greatest value.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The term “platform” (or “system”) may refer to an ensemble of: (i) instruments (e.g., imaging instruments, fluid controllers, temperature controllers, motion controllers and translation stages, etc.), (ii) devices (e.g., specimen slides, substrates, flow cells, microfluidic devices, etc., which may comprise fixed and/or removable or disposable components of the platform), (iii) reagents and/or reagent kits, and (iv) software, or any combination thereof, which allows a user to perform one or more bioassay methods (e.g., analyte detection, in situ detection or sequencing, and/or nucleic acid detection or sequencing) depending on the particular combination of instruments, devices, reagents, reagent kits, and/or software utilized.

The section headings used herein are for organizational purposes only and are not to be construed as limiting the subject matter described.

### Barcoding and Decoding Terminology:

A “barcode” is a label, or identifier, that conveys or is capable of conveying information (e.g., information about an analyte in a sample, a cell, a bead, a location, a sample, and/or a capture probe). The term “barcode” may refer either to a physical barcode molecule (e.g., a nucleic acid barcode molecule) or to its representation in a computer-readable, digital format (e.g., as a string of characters representing the sequence of bases in a nucleic acid barcode molecule).

The phrase “barcode diversity” refers to the total number of unique barcode sequences that may be represented by a given set of barcodes.

A physical barcode molecule (e.g., a nucleic acid barcode molecule) that forms a label or identifier as described above. In some instances, a barcode can be part of an analyte, can be independent of an analyte, can be attached to an analyte, or can be attached to or part of a probe that targets the analyte. In some instances, a particular barcode can be unique relative to other barcodes.

Physical barcodes can have a variety of different formats. For example, barcodes can include polynucleotide barcodes, random nucleic acid and/or amino acid sequences, and synthetic nucleic acid and/or amino acid sequences. A physical barcode can be attached to an analyte, or to another moiety or structure, in a reversible or irreversible manner. A physical barcode can be added to, for example, a fragment of a deoxyribonucleic acid (DNA) or ribonucleic acid (RNA) sample before or during sequencing of the sample. In some instances, barcodes can allow for identification and/or quantification of individual sequencing-reads in sequencing-based methods (e.g., a barcode can be or can include a unique molecular identifier or “UMI”). Barcodes can be used to detect and spatially-resolve molecular components found in biological samples, for example, at single-cell resolution (e.g., a barcode can be, or can include, a molecular barcode, a spatial barcode, a unique molecular identifier (UMI), etc.).

In some instances, barcodes may comprise a series of two or more segments or sub-barcodes (e.g., corresponding to “letters” or “code words” in a decoded barcode), each of which may comprise one or more of the subunits or building blocks used to synthesize the physical (e.g., nucleic acid) barcode molecules. For example, a nucleic acid barcode molecule may comprise two or more barcode segments, each of which comprises one or more nucleotides. In some instances, a barcode may comprise at least 1, 2, 3, 4, 5, 6, 7, 8, 9, 10, or more than 10 segments. In some instances, each segment of a barcode molecule may comprise at least 1, 2, 3, 4, 5, 6, 7, 8, 9, 10, 12, 14, 16, 18, 20, or more than 20 subunits or building blocks. For example, each segment of a nucleic acid barcode molecule may comprise at least 1, 2, 3, 4, 5, 6, 7, 8, 9, 10, 12, 14, 16, 18, 20, or more than 20 nucleotides. In some instances, two or more of the segments of a barcode may be separated by non-barcode segments, i.e., the segments of a barcode molecule need not be contiguous.

A “digital barcode” (or “digital barcode sequence”) is a representation of a corresponding physical barcode (or target analyte sequence) in a computer-readable, digital format as described above. A digital barcode may comprise one or more “letters” (e.g., 1, 2, 3, 4, 5, 6, 7, 8, 9, 10, 12, 14, 16, 18, 20, or more than 20 letters) or one or more “code words” (e.g., 1, 2, 3, 4, 5, 6, 7, 8, 9, 10, or more than 10 code words), where a “code word” comprises, e.g., 1, 2, 3, 4, 5, 6, 7, 8, 9, 10, 12, 14, 16, 18, 20, or more than 20 letters. In some instances, the sequence of letters or code words in a digital barcode sequence may correspond directly with the sequence of building blocks (e.g., nucleotides) in a physical barcode. In some instances, the sequence of letters or code words in a digital barcode sequence may not correspond directly with the sequence of building blocks in a physical barcode, but rather may comprise, e.g., arbitrary code words that each correspond to a segment of a physical barcode. For example, in some instances, the disclosed methods for decoding and error correction may be applied directly to detecting target analyte sequences (e.g., mRNA sequences) as opposed to detecting target barcodes, and the barcode probes used to detect the target analyte sequences may correspond to letters or code words that have been assigned to specific target analyte sequences but that do not directly correspond to the target analyte sequences.

A “designed barcode” (or “designed barcode sequence”) is a barcode (or its digital equivalent; in some instances a designed barcode may comprise a series of code words that can be assigned to gene transcripts and subsequently decoded into a decoded barcode) that meets a specified set of design criteria as required for a specific application. In some instances, a set of designed barcodes may comprise at least 2, at least 5, at least 10, at least 20, at least 40, at least 60, at least 80, at least 100, at least 200, at least 400, at least 600, at least 800, at least 1,000, at least 2,000, at least 4,000, at least 6,000, at least 8,000, at least 10,000, at least 20,000, at least 40,000, at least 60,000, at least 80,000, at least 100,000, at least 200,000, at least 400,000, at least 600,000, at least 800,000, at least 1,000,000, at least 2×106, at least 3×106, at least 4×106, at least 5×106, at least 6×106, at least 7×106, at least 8×106, at least 9×106, at least 107, at least 108, at least 109, or more than 109 unique barcodes. In some instances, a set of designed barcodes may comprise any number of designed barcodes within the range of values in this paragraph, e.g., 1,225 unique barcodes or 2.38×106 unique barcodes. As noted above for barcodes in general, in some instances designed barcodes may comprise two or more segments (corresponding to two or more code words in a decode barcode). In those cases, the specified set of design criteria may be applied to the designed barcodes as a whole, or to one or more segments (or positions) within the designed barcodes.

A “decoded barcode” (or “decoded barcode sequence”) is a digital barcode sequence generated via a decoding process that ideally matches a designed barcode sequence, but that may include errors arising from noise in the synthesis process used to create barcodes and/or noise in the decoding process itself. As noted above, in some instances, the disclosed methods for decoding and error correction may be applied directly to detecting target analytes (e.g., mRNA sequences) as opposed to detecting target barcodes, and the barcode probes used to detect the target analytes may correspond to letters or code words that have been assigned to specific target analytes but that do not directly correspond to the target analytes. In these instances, a decoded barcode (i.e., a series of letters or code words) may serve as a proxy for the target analyte.

A “corrected barcode” (or “corrected barcode sequence”) is a digital barcode sequence derived from a decoded barcode sequence by applying one or more error correction methods.

### Probe Terminology

The term “probe” may refer either to a physical probe molecule (e.g., a nucleic acid probe molecule) or to its representation in a computer-readable, digital format (e.g., as a string of characters representing the sequence of bases in a nucleic acid probe molecule). A “probe” may be, for example, a molecule designed to recognize (and bind or hybridize to) another molecule, e.g., a target analyte, another probe molecule, etc.

In some instances, a physical probe molecule may comprise one or more of the following: (i) a target recognition element (e.g., an antibody capable of recognizing and binding to a target peptide, protein, or small molecule; an oligonucleotide sequence that is complementary to a target gene sequence or gene transcript; or a poly-T oligonucleotide sequence that is complementary to the poly-A tails on messenger RNA molecules), (ii) a barcode element (e.g., a molecular barcode, a cell barcode, a spatial barcode, and/or a unique molecular identifier (UMI)), (iii) an amplification and/or sequencing primer binding site, (iv) one or more linker regions, (v) one or more detectable tags (e.g., fluorophores), or any combination thereof. In some instances, each component of a probe molecule may comprise at least 1, 2, 3, 4, 5, 6, 7, 8, 9, 10, 12, 14, 16, 18, 20, or more than 20 subunits or building blocks. For example, in some instances, each component of a nucleic acid probe molecule may comprise at least 1, 2, 3, 4, 5, 6, 7, 8, 9, 10, 12, 14, 16, 18, 20, or more than 20 nucleotides.

In some instances, physical probes may bind or hybridize directly to their target. In some instances, physical probes may bind or hybridize indirectly to their target. For example, in some instances, a secondary probe may bind or hybridize to a primary probe, where the primary probe binds or hybridizes directly to the target analyte. In some instances, a tertiary probe may bind or hybridize to a secondary probe, where the secondary probe binds or hybridizes to a primary probe, and where the primary probe binds or hybridizes directly to the target analyte.

Examples of “probes” and their applications include, but are not limited to, primary probes (e.g., molecules designed to recognize and bind or hybridize to target analyte), intermediate probes (e.g., molecules designed to recognize and bind or hybridize to another molecule and provide a hybridization or binding site for another probe (e.g., a detection probe), detection probes (e.g., molecules designed to recognize and bind or hybridize to another molecule, detection probes may be labeled with a fluorophore or other detectable tag). In some instances, a probe may be designed to recognize and bind (or hybridize) to a physical barcode sequence (or segments thereof). In some instances, a probe may be used to detect and decode a barcode, e.g., a nucleic acid barcode. In some instances, a probe may bind or hybridize directly to a target barcode. In some instances, a probe may bind or hybridize indirectly to a target barcode (e.g., by binding or hybridizing to other probe molecules which itself is bound or hybridized to the target barcode).

### Nucleic Acid Molecule and Nucleotide Terminology:

The terms “nucleic acid” (or “nucleic acid molecule”) and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include natural or non-natural nucleotides. In this regard, a naturally-occurring deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-natural bases that can be included in a nucleic acid or nucleotide are known in the art. See, for example, Appella (2009), “Non-Natural Nucleic Acids for Synthetic Biology”, Curr Opin Chem Biol. 13(5-6): 687-696; and Duffy, et al. (2020), “Modified Nucleic Acids: Replication, Evolution, and Next-Generation Therapeutics”, BMC Biology 18:112.

### Samples:

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some instances, the biological sample may comprise cells which are deposited on a surface.

Cell-free biological samples can include extracellular macromolecules, e.g., polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some instances, a substrate herein can be any support that is insoluble in aqueous liquid and which allows for positioning of biological samples, analytes, features, and/or reagents (e.g., probes) on the support. In some instances, a biological sample can be attached to a substrate. Attachment of the biological sample can be irreversible or reversible, depending upon the nature of the sample and subsequent steps in the analytical method. In certain instances, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. In some instances, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### Endogenous Analytes:

In some instances, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some instances, the analyte is inside a cell or on a cell surface, such as a transmembrane analyte or one that is attached to the cell membrane. In some instances, the analyte can be an organelle (e.g., nuclei or mitochondria). In some instances, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some instances described herein, an analyte may be a denatured nucleic acid, wherein the resulting denatured nucleic acid is single-stranded. The nucleic acid may be denatured, for example, optionally using formamide, heat, or both formamide and heat. In some instances, the nucleic acid is not denatured for use in a method disclosed herein.

In certain instances, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any implementation described herein, the analyte comprises a target sequence. In some instances, the target sequence may be endogenous to the sample, generated in the sample, added to the sample, or associated with an analyte in the sample. In some instances, the target sequence is a single-stranded target sequence (e.g., a sequence in a rolling circle amplification product). In some instances, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some instances, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

### Labelling Agents:

In some instances, provided herein are methods and compositions for analyzing endogenous analytes (e.g., RNA, ssDNA, and cell surface or intracellular proteins and/or metabolites) in a sample using one or more labelling agents. In some instances, an analyte labelling agent may include an agent that interacts with an analyte (e.g., an endogenous analyte in a sample). In some instances, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some instances, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some instances, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some instances, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some instances, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto.

Accordingly, terms such as “stain”, “staining”, “labeling”, and the like, may be used interchangeably to refer to elements, complexes, and macromolecules that allow a substance, structure, organelle, and/or component in a sample to be more easily detected than if said substance, structure, organelle, and/or component had not been stained or stained. For example, a tissue sample treated with a DNA dye such as DAPI (4′,6-diamidino-2-phenylindole) makes the nucleus of a cell more visible and makes detection or quantification of such cells easier than if they were not stained. Without being bound by theory or methodology, the labeling described herein may be used to mark a cell, structure, particle, or other target, and may be useful in discovering, determining expression, localization, confirmation, quantification, or measuring properties within a sample. Without limitation, labeling agents disclosed herein include stains, dyes, ligands, antibodies, particles, and other substances that may bind to or be localized at certain specific objects or locations. “Labels” or “labeling agents” may also refer to compounds or compositions which are conjugated or fused directly or indirectly to a reagent such as an oligonucleotide as disclosed herein or an antibody, and facilitates detection of the reagent to which it is conjugated or fused. The label may itself be detectable (e.g., radioisotope labels or fluorescent labels) or may catalyze chemical alteration of a substrate compound or composition which is detectable, e.g., an enzymatic label.

As provided by the invention disclosed herein, one or more features are derived by detecting nuclei, cell membrane, and/or cytoplasm of cells within the input image and/or by extracting features from the detected nuclei, cell membrane, and/or cytoplasm (depending upon the labeling agent(s) utilized within the input image). In some embodiments, features are derived by analyzing cell membrane staining, cell cytoplasm staining, and/or cell nucleus staining. Without being bound by theory or methodology “cytoplasmic staining” may describe a group of pixels arranged in a pattern bearing the morphological characteristics of a cytoplasmic region of a cell. Similarly “membrane staining” may refer to a group of pixels arranged in a pattern bearing the morphological characteristics of a cell membrane, preferably the plasma membrane separating the intracellular environment from the extracellular space; and “nucleus staining” may refer to a group of pixels with strong localized intensity in a pattern bearing the morphological characteristics of a nucleus of the cell. Those of skill in the art will appreciate that the nucleus, cytoplasm, and membrane of a cell have different characteristics and that differently stained tissue samples may reveal different biological features. For example, those of skill would understand that certain cell surface elements and receptors can have staining patterns localized to the membrane or localized to the cytoplasm. Thus, a “membrane” staining pattern may be analytically distinct from a “cytoplasmic” staining pattern. Likewise, a “cytoplasmic” staining pattern and a “nuclear” staining pattern may be analytically distinct.

In some such embodiments, labels or labelling comprises tissue and/or cell surface staining. Surface stains may include general lipid stains, fluorescent lipid analogues, sugar-binding lectins, label-conjugated protein-specific antibodies, and plasma membrane-specific dyes, stains, and label-conjugated antibodies. Those of skill in the art will appreciate and understand that a biological sample may be stained for different types of and/or cell membrane structures/components. Stains and dyes that label cell nuclei may include hematoxylin dyes, cyanine dyes, Draq dyes, and DAPI stain. Stains and dyes that label the cytoplasm of cells may include eosin dyes, fluorescein dyes, and the like. Alternatively, binding moieties (e.g., ligands, antibodies, and or peptides) directed/localizing to a cell membrane (e.g., the plasma membrane), the cytoplasm, the nucleus, or other structure/organelle of the cell may be conjugated to a labeling moiety described herein, thereby providing a detectable signal that identifies said membrane, cytoplasm, and/or nucleus. Such labeling can be used individually or in combination to aid in visualization, identification, and quantification of cells.

In some embodiments of the invention, the labelling described herein may be cell specific (e.g., cell-type specific), thus providing the detection of different cell types within a sample. In some embodiments, the invention disclosed herein, or elements thereof, incorporate identification of cell polarity and/or morphology. Cell polarity may refer to an asymmetry in molecular composition or structure between two sides, thus defining a polarity axis along which cellular processes will be differentially regulated. In some such embodiments, the invention incorporates identifying cellular symmetry, including the distribution of structures and/or organelles within the cells. For example and without limitation, the radial symmetry of labeled structures or organelles relative to other stains, e.g., plasma membrane, cytoplasmic and/or nuclear labels, such as the radial staining pattern of cytoskeletal structures or mitochondria relative to nuclear, cytoplasmic, and/or plasma membrane stains/labels in fibroblastic cell types. Similarly, the polarization of structures or organelles relative to other stains, e.g., plasma membrane, cytoplasmic and/or nuclear stains/labels, such as those polarized structures observed in the axonal projections of neuronal cells or the apical/basal polarity of epithelial cells.

Exemplary methods for staining tissue structures and guidance in the choice of stains appropriate for various purposes are known in the art and are discussed, for example, in “Sambrook et al., Molecular Cloning: A Laboratory Manual, Cold Spring Harbor Laboratory Press (1989)” and “Ausubel et al., Current Protocols in Molecular Biology, Greene Publishing Associates and Wiley-Intersciences (1987),” the disclosures of which are incorporated herein by reference. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some instances, an analyte binding moiety includes one or more antibodies or antigen binding fragments thereof. The antibodies or antigen binding fragments including the analyte binding moiety can specifically bind to a target analyte. In some instances, the analyte is a protein (e.g., a protein on a surface of the biological sample (e.g., a cell) or an intracellular protein). In some instances, a plurality of analyte labelling agents comprising a plurality of analyte binding moieties bind a plurality of analytes present in a biological sample. In some instances, the plurality of analytes includes a single species of analyte (e.g., a single species of polypeptide). In some instances in which the plurality of analytes includes a single species of analyte, the analyte binding moieties of the plurality of analyte labelling agents are the same. In some instances in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some instances, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some instances, the reporter oligonucleotides are releasable from the labelling agent, such as upon application of a stimulus. For example, the reporter oligonucleotide may be attached to the labelling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some instances, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### Assays for In Situ Detection and Analysis:

Objectives for in situ detection and analysis methods include detecting, quantifying, and/or mapping analytes (e.g., gene activity) to specific regions in a biological sample (e.g., a tissue sample or cells deposited on a surface) at cellular or sub-cellular resolution. Methods for performing in situ studies include a variety of techniques, e.g., in situ hybridization and in situ sequencing techniques. These techniques allow one to study the subcellular distribution of target analytes (e.g., gene activity as evidenced, e.g., by expressed gene transcripts), and have the potential to provide crucial insights in the fields of developmental biology, oncology, immunology, histology, etc.

Various methods can be used for in situ detection and analysis of target analytes, e.g., sequencing by synthesis (SBS), sequencing by ligation (SBL), sequencing by hybridization (SBH). Non-limiting examples of in situ hybridization techniques include single molecule fluorescence in situ hybridization (smFISH) and multiplexed error-robust fluorescence in situ hybridization (MERFISH). smFISH enables in situ detection and quantification of gene transcripts in tissue samples at the locations where they reside by making use of libraries of multiple short oligonucleotide probes (e.g., approximately 20 base pairs (bp) in length), each labeled with a fluorophore. The probes are sequentially hybridized to gene sequences (e.g., DNA) or gene transcript sequences (e.g., mRNA) sequences, and visualized as diffraction-limited spots by fluorescence microscopy (Levsky, et al. (2003) “Fluorescence In situ Hybridization: Past, Present and Future”, Journal of Cell Science 116(14):2833-2838; Raj, et al. (2008) “Imaging Individual mRNA Molecules Using Multiple Singly Labeled Probes”, Nat Methods 5(10): 877-879; Moor, et al. (2016), ibid.). Variations on the smFISH method include, for example, the use of combinatorial labelling schemes to improve multiplexing capability (Levsky, et al. (2003), ibid.), the use of smFISH in combination with super-resolution microscopy (Lubeck, et al. (2014) “Single-Cell In situ RNA Profiling by Sequential Hybridization”, Nature Methods 11(4):360-361).

MERFISH addresses two of the limitations of earlier in situ hybridization approaches, namely the limited number of target sequences that could be simultaneously identified and the robustness of the approach to readout errors caused by the stochastic nature of the hybridization process (Moor, et al. (2016), ibid.). MERFISH utilizes a binary barcoding scheme in which the probed target mRNA sequences are either fluorescence positive or fluorescence negative for any given imaging cycle (Ke, et al. (2016), ibid.; Moffitt, et al. (2016) “RNA Imaging with Multiplexed Error Robust Fluorescence In situ Hybridization”, Methods Enzymol. 572:1-49). The encoding probes that contain a combination of target-specific hybridization sequence regions and barcoded readout sequence regions are first hybridized to the target mRNA sequences. In each imaging cycle, a subset of fluorophore-conjugated readout probes is hybridized to a subset of encoding probes. Target mRNA sequences that fluoresce in a given cycle are assigned a value of “1” and the remaining target mRNA sequences are assigned a value of “0”. Between imaging cycles, the fluorescent probes from the previous cycle are photobleached. After, e.g., 14 or 16 rounds of readout probe hybridization and imaging, unique combinations of the detected fluorescence signals generate a 14-bit or 16-bit code that identifies the different gene transcripts. To address the increased error rate for correctly calling the readout codes increases as the number of hybridization and imaging cycles increases, the method may also entail the use of Hamming distances for barcode design and correction of decoding errors (see, e.g., Buschmann, et al. (2013) “Levenshtein Error-Correcting Barcodes for Multiplexed DNA Sequencing”, Bioinformatics 14:272), thereby resulting in an error-robust barcoding scheme.

Some in situ sequencing techniques generally comprise both in situ target capture (e.g., of mRNA sequences) and in situ sequencing. Non-limiting examples of in situ sequencing techniques include in situ sequencing with padlock probes (ISS-PLP), fluorescent in situ sequencing (FISSEQ), barcode in situ targeted sequencing (Barista-Seq), and spatially-resolved transcript amplicon readout mapping (STARmap) (see, e.g., Ke, et al. (2016), ibid., Asp, et al. (2020), ibid.).

Some methods for in situ detection and analysis of analytes utilize a probe (e.g., padlock or circular probe) that detects specific target analytes. The in situ sequencing using padlock probes (ISS-PLP) method, for example, combines padlock probing to target specific gene transcripts, rolling-circle amplification (RCA), and sequencing by ligation (SBL) chemistry. Within intact tissue sections, reverse transcription primers are hybridized to target sequence (e.g., mRNA sequences) and reverse transcription is performed to create cDNA to which a padlock probe (a single-stranded DNA molecule comprising regions that are complementary to the target cDNA) can bind (see, e.g., Asp, et al. (2020), ibid.). In one variation of the method, the padlock probe binds to the cDNA target with a gap remaining between the ends which is then filled in using a DNA polymerization reaction. In another variation of the method, the ends of the bound padlock probe are adjacent to each other. The ends are then ligated to create a circular DNA molecule. Target amplification using rolling-circle amplification (RCA) results in micrometer-sized RCA products (RCPs), containing a plurality of concatenated repeats of the probe sequence. In some examples, RCPs are then subjected to, e.g., sequencing-by-ligation (SBL) or sequencing-by-hybridization (SBH). In some cases, the method allows for a barcode located within the probe to be decoded.

Products of Endogenous Analytes and/or Labelling Agents:

In some instances, provided herein are methods and compositions for analyzing one or more products of an endogenous analyte and/or a labelling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some instances, a labelling agent that directly or indirectly binds to an analyte in the biological sample is analyzed. In some instances, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

In some instances, the analyzing comprises using primary probes which comprise a target binding region (e.g., a region that binds to a target such as RNA transcripts) and the primary probes may contain one or more barcodes (e.g., primary barcode). In some instances, the barcodes are bound by detection primary probes, which do not need to be fluorescent, but that include a target-binding portion (e.g., for hybridizing to one or more primary probes) and one or more barcodes (e.g., secondary barcodes). In some instances, the detection primary probe comprises an overhang that does not hybridize to the target nucleic acid but hybridizes to another probe. In some examples, the overhang comprises the barcode(s). In some instances, the barcodes of the detection primary probes are targeted by detectably labeled detection oligonucleotides, such as fluorescently labeled oligos. In some instances, one or more decoding schemes are used to decode the signals, such as fluorescence, for sequence determination. Various probes and probe sets can be used to hybridize to and detect an endogenous analyte and/or a sequence associated with a labelling agent. In some instances, these assays may enable multiplexed detection, signal amplification, combinatorial decoding, and error correction schemes. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set. The specific probe or probe set design can vary.

### Hybridization and Ligation:

Various probes and probe sets can be hybridized to an endogenous analyte and/or a labelling agent and each probe may comprise one or more barcode sequences. The specific probe or probe set design can vary. In some instances, the hybridization of a primary probe or probe set (e.g., a circularizable probe or probe set) to a target nucleic acid analyte and may lead to the generation of a rolling circle amplification (RCA) template. In some instances, the assay uses or generates a circular nucleic acid molecule which can be the RCA template.

In some instances, a product of an endogenous analyte and/or a labelling agent is a ligation product. In some instances, the ligation product is formed from circularization of a circularizable probe or probe set upon hybridization to a target sequence. In some instances, the ligation product is formed between two or more endogenous analytes. In some instances, the ligation product is formed between an endogenous analyte and a labelling agent. In some instances, the ligation product is formed between two or more labelling agent. In some instances, the ligation product is an intramolecular ligation of an endogenous analyte. In some instances, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some instances, provided herein is a probe or probe set capable of DNA-templated ligation, such as from a cDNA molecule. See, e.g., U.S. Pat. No. 8,551,710, which is hereby incorporated by reference in its entirety. In some instances, provided herein is a probe or probe set capable of RNA-templated ligation. See, e.g., U.S. Pat. Pub. 2020/0224244 which is hereby incorporated by reference in its entirety. In some instances, the probe set is a SNAIL probe set. See, e.g., U.S. Pat. Pub. 20190055594, which is hereby incorporated by reference in its entirety. In some instances, provided herein is a multiplexed proximity ligation assay. See, e.g., U.S. Pat. Pub. 20140194311 which is hereby incorporated by reference in its entirety. In some instances, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some instances, a circular probe can be indirectly hybridized to the target nucleic acid. In some instances, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some instances, the ligation involves chemical ligation. In some instances, the ligation involves template dependent ligation. In some instances, the ligation involves template independent ligation. In some instances, the ligation involves enzymatic ligation.

In some instances, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some instances, the ligase is a T4 RNA ligase. In some instances, the ligase is a splintR ligase. In some instances, the ligase is a single stranded DNA ligase. In some instances, the ligase is a T4 DNA ligase. In some instances, the ligase is a ligase that has an DNA-splinted DNA ligase activity. In some instances, the ligase is a ligase that has an RNA-splinted DNA ligase activity.

In some instances, the ligation herein is a direct ligation. In some instances, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some instances, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific implementations, the gap may be a gap of about 1, 2, 3, 4, 5, 6, 7, 8, 9 or 10 or more nucleotides, of any integer (or range of integers) of nucleotides in between the indicated values. In some instances, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some instances, the ligation herein is preceded by gap filling. In other implementations, the ligation herein does not require gap filling.

In some instances, ligation of the polynucleotides produces polynucleotides with melting temperature higher than that of un-ligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some instances, the ligation herein is a proximity ligation of ligating two (or more) nucleic acid sequences that are in proximity with each other, e.g., through enzymatic means (e.g., a ligase). In some instances, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### Primer Extension and Amplification:

In some instances, the hybridization of a primary probe or probe set (e.g., a circularizable probe or probe set) to a target analyte and may lead to the generation of an extension or amplification product. In some instances, a product is a primer extension product of an analyte, a labelling agent, a probe or probe set bound to the analyte (e.g., a circularizable probe bound to genomic DNA, mRNA, or cDNA), or a probe or probe set bound to the labelling agent (e.g., a circularizable probe bound to one or more reporter oligonucleotides from the same or different labelling agents.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, a product of an endogenous analyte and/or a labelling agent is an amplification product of one or more polynucleotides, for instance, a circular probe or circularizable probe or probe set. In some instances, the disclosed methods may comprise the use of a rolling circle amplification (RCA) technique to amplify signal. Rolling circle amplification is an isothermal, DNA polymerase-mediated process in which long single-stranded DNA molecules are synthesized on a short circular single-stranded DNA template using a single DNA primer (Zhao, et al. (2008), “Rolling Circle Amplification: Applications in Nanotechnology and Biodetection with Functional Nucleic Acids”, Angew Chem Int Ed Engl. 47(34):6330-6337; Ali, et al. (2014), “Rolling Circle Amplification: A Versatile Tool for Chemical Biology, Materials Science and Medicine”, Chem Soc Rev. 43(10):3324-3341). The RCA product is a concatemer containing tens to hundreds of tandem repeats that are complementary to the circular template, and may be used to develop sensitive techniques for the detection of a variety of targets, including nucleic acids (DNA, RNA), small molecules, proteins, and cells (Ali, et al. (2014), ibid.). In some implementations, a primer that hybridizes to the circular probe or circularized probe is added and used as such for amplification. In some instances, the RCA comprises a linear RCA, a branched RCA, a dendritic RCA, or any combination thereof.

In some instances, the amplification is performed at a temperature between or between about 20° C. and about 60° C. In some instances, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some instances, upon addition of a DNA polymerase in the presence of appropriate dNTP precursors and other cofactors, a primer is elongated to produce multiple copies of the circular template. This amplification step can utilize isothermal amplification or non-isothermal amplification. In some instances, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) are known in the art such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 November 15; 49(11): 2540-2550; Schweitzer et al. Proc. Natl Acad. Sci. USA 97:10113-119, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some instances, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some instances, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some instances, the RCA template may comprise the target analyte, or a part thereof, where the target analyte is a nucleic acid, or it may be provided or generated as a proxy, or a marker, for the analyte. In some instances, the RCA template may comprise a sequence of the probes and probe sets hybridized to an endogenous analyte and/or a labelling agent. In some instances, the amplification product can be generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (i.e., a reagent in a detection assay), which is used as a reporter for the assay, or a part of a reporter, or signal-generation system. The RCA template used to generate the RCP may thus be a circular (e.g., circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some instances, an assay may detect a product herein that includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein (e.g., a bridge probe or L-probe) may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe (e.g., a detection probe). The exogenously added nucleic acid probe may be optionally ligated to a cellular nucleic acid molecule or another exogenous nucleic acid molecule. In other examples, a product comprising a target sequence for a probe disclosed herein (e.g., an anchor probe) may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein (e.g., a bridge probe or L-probe) may be a probe hybridizing to an RCP. The probe may comprise an overhang that does not hybridize to the RCP but hybridizes to another probe (e.g., a detection probe).

### Signal Amplification Methods:

In some instances, a method disclosed herein may also comprise one or more signal amplification components and detecting such signals. In some instances, the present disclosure relates to the detection of nucleic acid sequences in situ using probe hybridization and generation of amplified signals associated with the probes. In some instances, the target nucleic acid of a nucleic acid probe comprises multiple target sequences for nucleic acid probe hybridization, such that the signal corresponding to a barcode sequence of the nucleic acid probe is amplified by the presence of multiple nucleic acid probes hybridized to the target nucleic acid. For example, multiple sequences can be selected from a target nucleic acid such as an mRNA, such that a group of nucleic acid probes (e.g., 20-50 nucleic acid probes) hybridize to the mRNA in a tiled fashion. In another example, the target nucleic acid can be an amplification product (e.g., an RCA product) comprising multiple copies of a target sequence (e.g., a barcode sequence of the RCA product).

Alternatively or additionally, amplification of a signal associated with a barcode sequence of a nucleic acid probe can be amplified using one or more signal amplification strategies off of an oligonucleotide probe that hybridizes to the barcode sequence. In some aspects, amplification of the signal associated with the oligonucleotide probe can reduce the number of nucleic acid probes needed to hybridize to the target nucleic acid to obtain a sufficient signal-to-noise ratio. For example, the number of nucleic acid probes to tile a target nucleic acid such as an mRNA can be reduced. In some aspects, reducing the number of nucleic acid probes tiling a target nucleic acid enables detection of shorter target nucleic acids, such as shorter mRNAs. In some instances, no more than one, two, three, 4, 5, 6, 7, 8, 9, 10, 11, 12, 13, 14, 15, 16, 17, 18. 19, or 20 nucleic acid probes may be hybridized to the target nucleic acid. In instances wherein the target nucleic acid is an amplification product, signal amplification off of the oligonucleotide probes may reduce the number of target sequences required for detection (e.g., the length of the RCA product can be reduced).

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some instances, a non-enzymatic signal amplification method may be used.

The detectable reactive molecules may comprise tyramide, such as used in tyramide signal amplification (TSA) or multiplexed catalyzed reporter deposition (CARD)-FISH. In some instances, the detectable reactive molecule may be releasable and/or cleavable from a detectable label such as a fluorophore. In some instances, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, WO 2019/236841, WO 2020/102094, WO 2020/163397, and WO 2021/067475, all of which are incorporated herein by reference in their entireties.

In some instances, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g., initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g., a sequence which is complementary to the exposed toehold region (input domain) of the first HCR monomers. In the absence of the HCR initiator, these interacting regions are protected by the secondary structure (e.g., they are not exposed), and thus the hairpin monomers are stable or kinetically trapped (also referred to as “metastable”), and remain as monomers (e.g., preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g., the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g., all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., WO 2020/123742 incorporated herein by reference), and may be used in the methods herein.

In some instances, similar to HCR reactions that use hairpin monomers, linear oligo hybridization chain reaction (LO-HCR) can also be used for signal amplification. In some instances, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some instances, the first species and/or the second species may not comprise a hairpin structure. In some instances, the plurality of LO-HCR monomers may not comprise a metastable secondary structure. In some instances, the LO-HCR polymer may not comprise a branched structure. In some instances, performing the linear oligo hybridization chain reaction comprises contacting the target nucleic acid molecule with the initiator to provide the initiator hybridized to the target nucleic acid molecule. In any of the instances herein, the target nucleic acid molecule and/or the analyte can be an RCA product.

In some instances, detection of nucleic acids sequences in situ includes combination of the sequential decoding methods described herein with an assembly for branched signal amplification. In some instances, the assembly complex comprises an amplifier hybridized directly or indirectly (via one or more oligonucleotides) to a sequence of an oligonucleotide probe described herein. In some instances, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some instances, the amplifier repeating sequence is about 5-30 nucleotides, and is repeated N times in the amplifier. In some instances, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some instances, an oligonucleotide probe described herein can be associated with an amplified signal by a method that comprises signal amplification by performing a primer exchange reaction (PER). In various instances, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various instances, the strand displacing polymerase is Bst. In various instances, the catalytic hairpin includes a stopper which releases the strand displacing polymerase. In various instances, branch migration displaces the extended primer, which can then dissociate. In various instances, the primer undergoes repeated cycles to form a concatemer primer (see e.g., U.S. Pat. Pub. No. US20190106733, which is incorporated herein by reference, for exemplary molecules and PER reaction components).

### Barcoded Analytes and Detection:

A target sequence for a probe disclosed herein may be comprised in any analyte disclose herein, including an endogenous analyte (e.g., a viral or cellular nucleic acid), a labelling agent, or a product generated in the biological sample using an endogenous analyte and/or a labelling agent.

In some aspects, one or more of the target sequences includes or is associated with one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode includes two or more sub-barcodes that together function as a single barcode. For example, a polynucleotide barcode can include two or more polynucleotide sequences (e.g., sub-barcodes) that are separated by one or more non-barcode sequences. In some instances, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding implementations, barcodes (e.g., primary and/or secondary barcode sequences) can be analyzed (e.g., detected or sequenced) using any suitable method or technique, including those described herein, such as sequencing by synthesis (SBS), sequencing by ligation (SBL), or sequencing by hybridization (SBH). In some instances, barcoding schemes and/or barcode detection schemes as described in RNA sequential probing of targets (RNA SPOTs), single-molecule fluorescent in situ hybridization (smFISH), multiplexed error-robust fluorescence in situ hybridization (MERFISH) or sequential fluorescence in situ hybridization (seqFISH+) can be used. In any of the preceding implementations, the methods provided herein can include analyzing the barcodes by sequential hybridization and detection with a plurality of labelled probes (e.g., detection probes (e.g., detection oligos) or barcode probes). In some instances, the barcode detection steps can be performed as described in hybridization-based in situ sequencing (HybISS). In some instances, probes can be detected and analyzed (e.g., detected or sequenced) as performed in fluorescent in situ sequencing (FISSEQ), or as performed in the detection steps of the spatially-resolved transcript amplicon readout mapping (STARmap) method. In some instances, signals associated with an analyte can be detected as performed in sequential fluorescent in situ hybridization (seqFISH).

In some instances, in a barcode-based detection method, barcode sequences are detected for identification of other molecules including nucleic acid molecules (DNA or RNA) longer than the barcode sequences themselves, as opposed to direct sequencing of the longer nucleic acid molecules. In some instances, a N-mer barcode sequence comprises 4N complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some instances, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and WO2019199579A1, which are hereby incorporated by reference in their entirety.

### Sequential Hybridization:

In some instances, the present disclosure relates to methods and compositions for encoding and detecting analytes in a temporally sequential manner for in situ analysis of an analyte in a biological sample, e.g., a target nucleic acid in a cell in an intact tissue. In some aspects, provided herein is a method for detecting the detectably-labeled probes, thereby generating a signal signature. In some instances, the signal signature corresponds to an analyte of the plurality of analytes. In some instances, the methods described herein are based, in part, on the development of a multiplexed biological assay and readout, in which a sample is first contacted with a plurality of nucleic acid probes comprising one or more probe types (e.g., labelling agent, circularizable probe, circular probe, etc.), allowing the probes to directly or indirectly bind target analytes, which may then be optically detected (e.g., by detectably-labeled probes) in a temporally-sequential manner. In some instances, the probes or probe sets comprising various probe types may be applied to a sample simultaneously. In some instances, the probes or probe sets comprising various probe types may be applied to a sample sequentially. In some aspects, the method comprises sequential hybridization of labelled probes to create a spatiotemporal signal signature or code that identifies the analyte.

In some aspects, provided herein is a method involving a multiplexed biological assay and readout, in which a sample is first contacted with a plurality of nucleic acid probes, allowing the probes to directly or indirectly bind target analytes, which may then be optically detected (e.g., by detectably-labeled probes) in a temporally sequential manner. The plurality of nucleic acid probes themselves may be detectably-labeled and detected; in other words, the nucleic acid probes themselves serve as the detection probes. In other implementations, a nucleic acid probe itself is not directly detectably-labeled (e.g., the probe itself is not conjugated to a detectable label); rather, in addition to a target binding sequence (e.g., a sequence binding to a barcode sequence in an RCA product), the nucleic acid probe further comprises a sequence for detection which can be recognized by one or more detectably-labeled detection probes. In some instances, the probes or probe sets comprising various probe types may be applied to a sample simultaneously. In some instances, the probes or probe sets comprising various probe types may be applied to a sample sequentially. In some instances, the method comprises detecting a plurality of analytes in a sample.

In some instances, the method presented herein comprises contacting the sample with a plurality of probes comprising one or more probes having distinct labels and detecting signals from the plurality of probes in a temporally sequential manner, wherein said detection generates signal signatures each comprising a temporal order of signal or absence thereof, and the signal signatures correspond to said plurality of probes that identify the corresponding analytes. In some instances, the temporal order of the signals or absence thereof corresponding to the analytes can be unique for each different analyte of interest in the sample. In some instances, the plurality of probes hybridize to an endogenous molecule in the sample, such as a cellular nucleic acid molecule, e.g., genomic DNA, RNA (e.g., mRNA), or cDNA. In some instances, the plurality of probes hybridize to a product of an endogenous molecule in the sample (e.g., directly or indirectly via an intermediate probe). In some instances, the plurality of probes hybridize to labelling agent that binds directly or indirectly to an endogenous molecule in the sample or a product thereof. In some instances, the plurality of probes hybridize to a product (e.g., an RCA product) of a labelling agent that binds directly or indirectly to an endogenous molecule in the sample or a product thereof.

In any of the implementations disclosed herein, the detection of signals can be performed sequentially in cycles, one for each distinct label. In any of the implementations disclosed herein, signals or absence thereof from detectably-labeled probes targeting an analyte in a particular location in the sample can be recorded in a first cycle for detecting a first label, and signals or absence thereof from detectably-labeled probes targeting the analyte in the particular location can be recorded in a second cycle for detecting a second label distinct from the first label. In any of the implementations disclosed herein, a unique signal signature can be generated for each analyte of the plurality of analytes. In any of the implementations disclosed herein, one or more molecules comprising the same analyte or a portion thereof can be associated with the same signal signature.

In some instances, the in situ assays employ strategies for optically encoding the spatial location of target analytes (e.g., mRNAs) in a sample using sequential rounds of fluorescent hybridization. Microcopy may be used to analyze 4 or 5 fluorescent colors indicative of the spatial localization of a target, followed by various rounds of hybridization and stripping, in order to generate a large set of unique optical signal signatures assigned to different analytes. These methods often require a large number of hybridization rounds, and a large number of microscope lasers (e.g., detection channels) to detect a large number of fluorophores, resulting in a one to one mapping of the lasers to the fluorophores. Specifically, each detectably-labeled probe comprises one detectable moiety, e.g., a fluorophore.

In some aspects, provided herein is a method for analyzing a sample using a detectably-labeled set of probes. In some instances, the method comprises contacting the sample with a first plurality of detectably-labeled probes for targeting a plurality of analytes; performing a first detection round comprising detecting signals from the first plurality of detectably-labeled probes; contacting the sample with a second plurality of detectably-labeled probes for targeting the plurality of analytes; performing a second detection round of detecting signals from the second plurality of detectably-labeled probes, thereby generating a signal signature comprising a plurality of signals detected from the first detection round and second detection round, wherein the signal signature corresponds to an analyte of the plurality of analytes.

In some instances, detection of an optical signal signature comprises several rounds of detectably-labeled probe hybridization (e.g., contacting a sample with detectably-labeled probes), detectably-labeled probe detection, and detectably-labeled probe removal. In some instances, a sample is contacted with plurality first detectably-labeled probes, and said probes are hybridized to a plurality of nucleic acid analytes within the sample in decoding hybridization round 1. In some instances, a first detection round is performed following detectably-labeled probe hybridization. After hybridization and detection of a first plurality of detectably-labeled probes, probes are removed, and a sample may be contacted with a second plurality round of detectably-labeled probes targeting the analytes targeted in decoding hybridization round 1. The second plurality of detectably-labeled probes may hybridize to the same nucleic acid(s) as the first plurality of detectably-labeled probes (e.g., hybridize to an identical or hybridize to new nucleic acid sequence within the same nucleic acid), or the second plurality of detectably-labeled probes may hybridize to different nucleic acid(s) compared to the first plurality of detectably-labeled probes. Following m rounds of contacting a sample with a plurality of detectably-labeled probes, probe detection, and probe removal, ultimately a unique signal signature to each nucleic acid is produced that may be used to identify and quantify said nucleic acids and the corresponding analytes (e.g., if the nucleic acids themselves are not the analytes of interest and each is used as part of a labelling agent for one or more other analytes such as protein analytes and/or other nucleic acid analytes).

In some instances, after hybridization of a detectably-labeled probes (e.g., fluorescently labeled oligonucleotide) that detects a sequence (e.g., barcode sequence on a secondary probe or a primary probe), and optionally washing away the unbound molecules of the detectably-labeled probe, the sample is imaged and the detection oligonucleotide or detectable label is inactivated and/or removed. In some instances, removal of the signal associated with the hybridization between rounds can be performed by washing, heating, stripping, enzymatic digestion, photo-bleaching, displacement (e.g., displacement of detectably-labeled probes with another reagent or nucleic acid sequence), cleavage, quenching, chemical degradation, bleaching, oxidation, or any combinations thereof.

In some examples, removal of a probe (e.g., un-hybridizing the entire probe), signal modifications (e.g., quenching, masking, photo-bleaching, signal enhancement (e.g., via FRET), signal amplification, etc.), signal removal (e.g., cleaving off or permanently inactivating a detectable label) can be performed. Inactivation may be caused by removal of the detectable label (e.g., from the sample, or from the probe, etc.), and/or by chemically altering the detectable label in some fashion, e.g., by photobleaching the detectable label, bleaching or chemically altering the structure of the detectable label, e.g., by reduction, etc.). In some instances, the fluorescently labeled oligonucleotide and/or the intermediate probe hybridized to the fluorescently labeled oligonucleotide (e.g., bridge probe or L-probe) can be removed. In some instances, a fluorescent detectable label may be inactivated by chemical or optical techniques such as oxidation, photobleaching, chemically bleaching, stringent washing or enzymatic digestion or reaction by exposure to an enzyme, dissociating the detectable label from other components (e.g., a probe), chemical reaction of the detectable label (e.g., to a reactant able to alter the structure of the detectable label) or the like. For instance, bleaching may occur by exposure to oxygen, reducing agents, or the detectable label could be chemically cleaved from the nucleic acid probe and washed away via fluid flow.

In some instances, removal of a signal comprises displacement of probes with another reagent (e.g., probe) or nucleic acid sequence. For example, a given probe (e.g., detectably-labeled probes and/or the intermediate probe hybridized to the fluorescently labeled oligonucleotide (e.g., bridge probe or L-probe)) may be displaced by a subsequent probe that hybridizes to an overlapping region shared between the binding sites of the probes. In some cases, a displacement reaction can be very efficient, and thus allows for probes to be switched quickly between cycles, without the need for chemical stripping (or any of the damage to the sample that is associated therewith). In some instances, a sequence for hybridizing the subsequent or displacer probe (i.e., a toehold sequence) may be common across a plurality of probes capable of hybridizing to a given binding site. In some aspects, a single displacement probe can be used to simultaneously displace detection probes bound to an equivalent barcode position from all of the RCPs within a given sample simultaneously (with the displacement mediated by the subsequent detection probes). This may further increase efficiency and reduce the cost of the method, as fewer different probes are required.

After a signal is inactivated and/or removed, then the sample is re-hybridized in a subsequent round with a subsequent fluorescently labeled oligonucleotide, and the oligonucleotide can be labeled with the same color or a different color as the fluorescently labeled oligonucleotide of the previous cycle. In some instances, as the positions of the analytes, probes, and/or products thereof can be fixed (e.g., via fixing and/or crosslinking) in a sample, the fluorescent spot corresponding to an analyte, probe, or product thereof remains in place during multiple rounds of hybridization and can be aligned to read out a string of signals associated with each target analyte.

### Decoding:

A “decoding process” is a process comprising a plurality of decoding cycles in which different sets of barcode probes are contacted with target analytes (e.g., mRNA sequences) or target barcodes (e.g., barcodes associated with target analytes) present in a sample, and used to detect the target sequences or associated target barcodes, or segments thereof. In some instances, the decoding process comprises acquiring one or more images (e.g., fluorescence images) for each decoding cycle. Decoded barcode sequences are then inferred based on a set of physical signals (e.g., fluorescence signals) detected in each decoding cycle of a decoding process. In some instances, the set of physical signals (e.g., fluorescence signals) detected in a series of decoding cycles for a given target barcode (or target analyte sequence) may be considered a “signal signature” for the target barcode (or target analyte sequence). In some instances, a decoding process may comprise, e.g., 1, 2, 3, 4, 5, 6, 7, 8, 9, 10, or more than 10 decoding cycles. In some instances, each decoding cycle may comprise contacting a plurality of target sequences or target barcodes with 1, 2, 3, 4, 5, 6, 7, 8, 9, 10, or more than 10 barcode probes (e.g., fluorescently-labeled barcode probes) that are configured to hybridize or bind to specific target sequences or target barcodes, or segments thereof. In some instances, a decoding process may comprise performing a series of in situ barcode probe hybridization steps and acquiring images (e.g., fluorescence images) at each step. Systems and methods for performing multiplexed fluorescence in situ hybridization and imaging are described in, for example, WO 2021/127019 A1; U.S. Pat. No. 11,021,737; and PCT/EP2020/065090 (WO2020240025A1), each of which is incorporated herein by reference in its entirety.

### Anchor Probes:

In some instances, the present methods may further involve contacting the target analyte, e.g., a nucleic acid molecule, or proxy thereof with an anchor probe. In some instances, the anchor probe comprises a sequence complementary to an anchor probe binding region, which is present in all target nucleic acid molecules (e.g., in primary or secondary probes), and a detectable label. The detection of the anchor probe via the detectable label confirms the presence of the target nucleic acid molecule. The target nucleic acid molecule may be contacted with the anchor probe prior to, concurrently with, or after being contacted with the first set of detection probes. In some instances, the target nucleic acid molecule may be contacted with the anchor probe during multiple decoding cycles. In some instances, multiple different anchor probes comprising different sequences and/or different reporters may be used to confirm the presence of multiple different target nucleic acid molecules. The use of multiple anchor probes is particularly useful when detection of a large number of target nucleic acid molecules is required, as it allows for optical crowding to be reduced and thus for detected target nucleic acid molecules to be more clearly resolved.

### Analysis of a Biological Sample and Optics Module of an Imaging System

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Target molecules (e.g., nucleic acids, proteins, antibodies, etc.) can be detected in biological samples (e.g., one or more cells or a tissue sample) using an instrument having integrated optics and fluidics modules (an “opto-fluidic instrument” or “opto-fluidic system”). In an opto-fluidic instrument, the fluidics module is configured to deliver one or more reagents (e.g., fluorescent probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles. In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

A sample disclosed herein can be or be derived from any biological sample. Biological samples may be obtained from any suitable source using any of a variety of techniques including, but not limited to, biopsy, surgery, and laser capture microscopy (LCM), and generally includes cells, tissues, and/or other biological material from the subject. A biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample from a mammal. A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic subjects, subjects that have or are suspected of having a disease (e.g., an individual with a disease such as cancer) or a pre-disposition to a disease, and/or subjects in need of therapy or suspected of needing therapy.

The biological sample can include any number of macromolecules, for example, cellular macromolecules and organelles (e.g., mitochondria and nuclei). The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

In some embodiments, the biological sample may comprise cells or a tissue sample which are deposited on a substrate. As described herein, a substrate can be any support that is insoluble in aqueous liquid and allows for positioning of biological samples, analytes, features, and/or reagents on the support. In some embodiments, a biological sample is attached to a substrate. In some embodiments, the substrate is optically transparent to facilitate analysis on the opto-fluidic instruments disclosed herein. For example, in some instances, the substrate is a glass substrate (e.g., a microscopy slide, cover slip, or other glass substrate). Attachment of the biological sample can be irreversible or reversible, depending upon the nature of the sample and subsequent steps in the analytical method. In certain embodiments, the sample can be attached to the substrate reversibly by applying a suitable polymer coating to the substrate and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

### PENULTIMATE COMMENTS

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described but can be practiced with modification and alteration within the scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A system, comprising:
an illumination dichroic mirror;
a light source configured to generate illumination light and direct the illumination light to the illumination dichroic mirror;
the illumination dichroic mirror being configured to direct at least a portion of the illumination light from the light source to an objective;
the objective disposed to direct the illumination light to a sample positioned in the field of view of the objective;
an autofocus system comprising an autofocus light source configured to generate reference light; and
an image sensor configured to receive, via the objective, a reflection of the reference light from a reflective interface in the field of view of the objective,
wherein:
the autofocus system is configured to direct the reference light from the autofocus light source to the illumination dichroic mirror such that the reference light is incident on the illumination dichroic mirror parallel to the illumination light, and
the illumination dichroic mirror is further configured to direct at least a portion of the reference light to the objective.

2. The system of claim 1, wherein the autofocus system further comprises a reference dichroic mirror configured to direct the reference light from the autofocus light source to the illumination dichroic mirror by reflecting at least a portion of the reference light.

3. The system of claim 2, wherein the reference dichroic mirror is further configured to transmit at least a portion of the illumination light.

4. The system of claim 1 further comprising a condenser disposed to direct the illumination light to the illumination dichroic mirror and/or to direct the reference light from the reference dichroic mirror to the illumination dichroic mirror.

5. The system of claim 4, wherein the condenser is disposed to focus the illumination light to a back focal plane of the objective and the reference dichroic mirror is further configured to direct the reference light to the condenser at an oblique angle to the collimation axis.

6. The system of claim 1, further comprising an additional image sensor configured to receive, via the objective, emission light emitted by the sample.

7. The system of claim 1, wherein the image sensor is further configured to receive, via the objective, emission light emitted by the sample.

8. The system of 7, wherein the system further comprises a tube lens disposed to focus the emission light from the sample onto the image sensor.

9. The system of claim 8, wherein the objective is disposed to direct the emission light from the sample to the tube lens.

10. The system of claim 8, wherein the illumination dichroic mirror is further configured to transmit the emission light and the reflection of the reference light from the sample to the tube lens, and the tube lens is configured to focus the transmitted reference light onto the image sensor.

11. The system of claim 7, further comprising a filter disposed between the objective focal plane and the image sensor, the filter configured to transmit at least a portion of the emission light and at least a portion of the reference light.

12. The system of claim 1, further comprising one or more processors configured to: identify a detected position of the reflection of the reference light on the image sensor, and identify a difference between the detected position and a reference position on the image sensor.

13. The system of claim 1, wherein the reference light is spatially coherent.

14. The system of claim 1, wherein the autofocus light source comprises a laser, an LED projector or a superluminescent diode.

15. The system of claim 1, further comprising an illuminator module comprising the light source and the autofocus system.

16. The system of claim 15, wherein the illuminator module is configured to change a wavelength of the illumination light by moving the light source, and the autofocus system is configured to be stationary during movement of the light source.

17. The system of claim 1, further comprising one or more processors configured to detect a characteristic of the reflection of the reference light received by the image sensor to determine, based on the characteristic, a position of the sample relative to the objective focal plane.

18. The system of claim 17, wherein the characteristic is a position of the reference light on the image sensor.

19. The system of claim 1, wherein the system is an imaging system.
